(19) United States
(12) Patent Application Publication
(10) Pub. No.: US 2020/0254118 A1
(43) Pub. Date: Aug. 13, 2020

(54) BREAST CANCER DETECTION USING B7-H3-TARGETED MOLECULAR IMAGING

(71) Applicants: Sunitha Bachawal, Santa Clara, CA (US); Juergen Karl Willmann, Stanford, CA (US)

(72) Inventors: Sunitha Bachawal, Santa Clara, CA (US); Juergen Karl Willmann, Stanford, CA (US)

(21) Appl. No.: 15/776,351

(22) PCT Filed: Dec. 2, 2016
(86) PCT No.: PCT/US2016/064572

§ 371 Date: May 15, 2018

(51) Int. Cl. A61K 49/22; C07K 16/28; C07K 16/30; G01N 33/574; A61P 35/00; A61P 31/00

(52) U.S. Cl. CPC A61K 49/221; C07K 16/2827; C07K 16/3015; A61K 45/06; A61P 35/00; A61P 31/00; G01N 33/57415

(57) ABSTRACT

Compositions and methods for detection and diagnosis of breast cancer are disclosed. In particular, the invention relates to the use of B7-H3-targeted imaging agents for molecular imaging of breast cancer. Vascular expression of B7-H3 is selectively and significantly higher in breast cancer tissues. Detection of B7-H3 can be used to distinguish between benign and malignant lesions with high diagnostic accuracy. B7-H3-targeted imaging agents can be produced by conjugation of a B7-H3-binding antibody, aptamer, or other ligand to various diagnostic agents, such as contrast agents, photoactive agents, or detectable labels that are useful for detection and medical imaging of breast cancer tumors.

### STATEMENT REGARDING FEDERALLY SPONSORED RESEARCH OR DEVELOPMENT

This invention was made with Government support under contract CA155289 awarded by the National Institutes of Health. The Government has certain rights in the invention.

### TECHNICAL FIELD

The present invention pertains generally to targeted molecular imaging of breast cancer. In particular, the invention relates to imaging agents comprising an anti-B7 homolog 3 (B7-H3) targeting agent, such as a B7-H3-binding antibody, aptamer, or other ligand conjugated to a diagnostic or detection agent that can be used to detect breast cancer and distinguish benign and malignant lesions. Additionally, the B7-H3 targeting agent may further be conjugated to a therapeutic agent for treating breast cancer.

### BACKGROUND

Breast cancer is the second leading cause of cancer-related deaths in women in the United States, with an estimated 232,670 new diagnoses and 40,000 deaths from this cancer in 2014 (Siegel et al. (2014) CA Cancer J. Clin. 64:9-29). If detected early, survival of women with breast cancer can be substantially increased compared with detection at later stages. The 5-year survival rate in patients diagnosed with stage I and II disease is 100% and 98.5% compared with 84.6% and 25.0, respectively, when detected at stage III and IV disease (Siegel et al., supra). Next to breast self-exam and clinical breast exam, the American Cancer Society recommends mammography as a screening exam in women ages 40 years and older (Smith et al. (2014) CA Cancer J. Clin. 64:30-51). For high-risk women, mammography is recommended at age 30 years (Smith et al., supra).

However, the presence of dense or heterogeneously dense breast tissue, which is particularly prevalent in younger patients (Wang et al. (2014) Mayo Clin. Proc. 89:548-857), may decrease diagnostic accuracy of mammography in detecting breast cancer, with sensitivities ranging between 30% and 55% (Berg et al. (2012) JAMA 307:1394-1404; Boyd et al. (2007) N. Engl. J. Med. 356:227-236; Kolb et al. (2002) Radiology 225:165-175). Adding ultrasound to screening mammography is currently being explored as a complementary screening approach for earlier breast cancer detection in women with dense breast tissue (Scheel et al. (2015) Am. J. Obstet. Gynecol. 212:9-17). Several studies have addressed the value of adding breast ultrasound imaging to screening mammography and demonstrated an increase in cancer detection rates ranging from 0.3 to 7.7 cancers per 1,000 women screened (Kolb et al., supra; Scheel et al., supra; Buchberger et al. (2000) Semin. Ultrasound CT MR 21:325-336; Leconte et al. (2003) AJR Am. J. Roentgenol. 180:1675-1679; Kaplan et al. (2001) Radiology 221:641-649; Berg et al. (2008) JAMA 299:2151-2163). Berg and colleagues showed that breast cancer was diagnosed on ultrasound alone in 12 of 40 patients (30%; Berg et al., supra). However, the diagnostic accuracy of current ultrasound screening techniques in breast cancer detection is low with a positive predictive value as low as 8.6% (Berg et al., supra) or even lower 5.6% in another study (Hooley et al. (2012) Radiology 265:59-69), resulting in a large number of unnecessary callbacks and biopsies. In addition, the sensitivity of ultrasound performed alone in detecting invasive breast cancer was only 50% (Berg et al., supra) and 27% in another prospective multimodality screening study (Weinstein et al. (2009) J. Clin. Oncol. 27:6124-6128). Therefore, further improvement of the diagnostic accuracy of ultrasound imaging is critically needed for women enrolled in breast cancer screening.

Thus, there remains a need for better methods for diagnosing breast cancer that can distinguish normal, benign, precancerous, and malignant breast lesions and enable early detection.

### SUMMARY

The present invention relates to B7-H3-targeted imaging of breast cancer. B7-H3-targeted imaging agents can be used to detect overexpression of B7-H3 on breast tumor neovasculature, breast tumor epithelium, cancerous cells, or precancerous lesions and allows differentiation of benign, premalignant, and malignant lesions.

In one aspect, the invention includes a method of detecting breast cancer, the method comprising: a) administering a detectably effective amount of a B7-H3-targeted imaging agent to a patient suspected or at risk of having breast cancer, under conditions wherein the B7-H3-targeted imaging agent binds to B7-H3 on breast tumor neovasculature, breast tumor epithelium, cancerous cells, or precancerous lesions, if present, in the patient; and b) detecting the B7-H3-targeted imaging agent bound to the breast tumor neovasculature, breast tumor epithelium, cancerous cells, or precancerous lesions, if present, by imaging breast tissue of the patient. In certain embodiments, the breast cancer is luminal A breast cancer, luminal B breast cancer, triple negative breast cancer, or Her2-positive breast cancer.

In certain embodiments, imaging of breast tissue is performed using a method selected from the group consisting of ultrasound imaging (UI), positron emission tomography (PET), single photon emission computed tomography (SPECT), magnetic resonance imaging (MRI), computed tomography (CT), optical imaging (Op, photoacoustic imaging (PI), fluoroscopy, and fluorescence imaging.

In certain embodiments, the B7-H3-targeting agent is an antibody, peptide, peptoid, aptamer, small molecule ligand, or any other agent that selectively binds to B7-H3.

In certain embodiments, the B7-H3-targeted imaging agent comprises an anti-B7-H3 antibody conjugated to a diagnostic agent. The anti-B7-H3 antibody may be a monoclonal antibody, a polyclonal antibody, a chimeric antibody, a recombinant fragment of an antibody, a Fab fragment, a Fab′ fragment, a F(ab)₂ fragment, a Fᵥ fragment, or an scFᵥ fragment.

In other embodiments, the B7-H3-targeted imaging agent comprises an aptamer conjugated to a diagnostic agent. The aptamer may be a nucleic acid aptamer or a peptide aptamer.

The diagnostic agent can be, for example, a contrast agent (e.g., ultrasound contrast agent, a magnetic resonance imaging (MRI) contrast agent, or a radiocontrast agent), an isotopic label, a fluorescent label, a chemiluminescent label, a bioluminescent label, a paramagnetic ion, an enzyme, or a photoactive agent.

Exemplary contrast agents include ultrasound contrast agents (e.g. microbubbles), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate).

Exemplary fluorescent labels include fluorescein derivatives, rhodamine derivatives, coumarin derivatives, cyanine derivatives, acridine derivatives, squaraine derivatives, naphthalene derivatives, oxadiazol derivatives, anthracene derivatives, pyrene derivatives, oxazine derivatives, arylmethine derivatives, and tetrapyrrole derivatives. In addition, the fluorescent label may comprise a fluorescent protein, such as, but not limited to, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, rsCherry, and rsCherryRev.

Exemplary isotopic labels may comprise radioactive isotopes (e.g., gamma-emitters, beta-emitters, and positron-emitters) or non-radioactive isotopes (e.g., stable trace isotopes), such as, but not limited to, ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴CU, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr.

Exemplary paramagnetic ions include chromium (III), manganese (II), iron (III), iron (II), cobalt (II), nickel (II), copper (II), neodymium (III), samarium (III), ytterbium (III), gadolinium (III), vanadium (II), terbium (III), dysprosium (III), holmium (III) and erbium (III).

The methods of the invention can be used for determining the prognosis of the patient. For example, detection of a precancerous lesion indicates the patient is at risk of developing breast cancer. Detection of increased levels of B7-H3 antigen on the surface of breast tumor neovasculature, breast tumor epithelium, or cancerous cells is associated with tumor growth and cancer progression.

In another aspect, the invention includes a method of performing image-guided surgery on breast tissue of a patient having breast cancer, the method comprising: a) detecting breast cancer according to a method described herein; and b) using at least one image of the breast tissue to determine where resection of cancerous tissue is needed. Imaging of the breast tissue may be performed, for example, pre-operatively to assist surgical planning and/or intra-operatively to provide image-guidance during surgery, e.g., for tumor margin delineation or evaluation of completeness of resection.

In certain embodiments, the imaging is performed with a medical imaging device selected from the group consisting of an ultrasound scanner, a magnetic resonance imaging instrument, a radiography system, an X-ray computed tomography (CT) scanner, a computed axial tomography (CAT) scanner, a gamma camera, and a positron emission tomography (PET) scanner.

In certain embodiments, the medical imaging device is a miniaturized medical imaging system. For example, the miniaturized imaging system may comprise a handheld microscope, a laparoscope, an endoscope, or a microendoscope comprising an imaging system. In certain embodiments, the medical imaging device is an intravascular ultrasound (IVUS) imaging device or a fluorescence imaging device.

In another aspect, the invention includes a method of imaging breast tissue of a patient suspected or at risk of having breast cancer, the method comprising: a) contacting breast tissue of the patient with a detectably effective amount of a B7-H3-targeted imaging agent under conditions wherein the B7-H3-targeted imaging agent binds to B7-H3 on any breast tumor neovasculature, breast tumor epithelium, cancerous cells, or precancerous lesions, if present, in the breast tissue; and b) imaging breast tissue of the patient, wherein detection of increased binding of the B7-H3-targeted imaging agent to the breast tissue of the patient compared to a control indicates that the patient has breast cancer. The breast tissue may be contacted with the B7-H3-targeted imaging agent either in vivo or in vitro. In certain embodiments, imaging of breast tissue is performed using a method selected from the group consisting of ultrasound imaging (UI), positron emission tomography (PET), single photon emission computed tomography (SPECT), magnetic resonance imaging (MRI), computed tomography (CT), optical imaging (OI) photoacoustic imaging (PI), fluoroscopy, and fluorescence imaging.

In another aspect, the invention includes a method of monitoring progression of breast cancer in a patient, the method comprising: imaging breast tissue of the patient according to a method described herein, wherein a first image is obtained at a first time point and a second image is obtained later at a second time point, wherein detection of increased binding of the B7-H3-targeted imaging agent to the breast tissue of the patient at the second time point compared to the first time point indicates that the patient is worsening, and detection of decreased binding of the B7-H3-targeted imaging agent to the breast tissue of the patient at the second time point compared to the first time point indicates that the patient is improving. Increased binding of the B7-H3-targeted imaging agent to the breast tissue of the patient may be caused, for example, by growth of a breast cancer tumor or the presence of more breast cancer tumors or lesions at the second time point, which can be determined by inspection of the images. Alternatively, decreased binding of the B7-H3-targeted imaging agent to the breast tissue of the patient may be caused, for example, by tumor shrinkage or the presence of fewer breast cancer tumors or lesions.

In another aspect, the invention includes a method for evaluating the effect of an agent for treating breast cancer in a patient, the method comprising: imaging breast tissue of the patient according to a method described herein before and after the patient is treated with the agent, wherein detection of increased binding of the B7-H3-targeted imaging agent to the breast tissue of the patient (e.g., from tumor growth or increase in number of tumors or cancer cells) after the patient is treated with the agent compared to before the patient is treated with the agent indicates that the patient is worsening, and decreased binding of the B7-H3-targeted imaging agent to the breast tissue of the patient (e.g., from reduction in tumor size or reduction in the number of cancer cells) after the subject is treated with the agent compared to before the patient is treated with the agent indicates that the patient is improving.

A method for monitoring the efficacy of a therapy for treating breast cancer in a patient, the method comprising: imaging breast tissue of the patient according to a method described herein before and after the subject undergoes the therapy, wherein detection of increased binding of the B7-H3-targeted imaging agent to the breast tissue of the patient (e.g., from tumor growth or increase in number of tumors or cancer cells) after the patient undergoes the therapy compared to before the patient undergoes the therapy indicates that the patient is worsening, and decreased binding of the B7-H3-targeted imaging agent to the breast tissue of the patient (e.g., from reduction in tumor size or reduction in the number of cancer cells) after the patient undergoes the therapy compared to before the patient undergoes the therapy indicates that the patient is improving.

In another aspect, the invention includes a kit comprising an anti-B7-H3 antibody, described herein, or a bioconjugate thereof (e.g., B7-H3-targeted imaging agent, therapeutic agent, or theranostic agent) and instructions for using the kit to diagnose and/or treat breast cancer. In one embodiment, the kit comprises an ultrasound contrast agent comprising an anti-B7-H3 antibody conjugated to a microbubble and instructions for performing ultrasound.

In another aspect, the invention includes a method of treating a patient suspected or at risk of having breast cancer, the method comprising: a) receiving information regarding whether or not breast cancer was detected in the patient using a B7-H3-targeted imaging agent according to a method described herein; and b) administering anti-cancer therapy to the subject if breast cancer was detected in the patient. In certain embodiments, the anti-cancer therapy comprises surgery, radiation therapy, chemotherapy, hormonal therapy, immunotherapy, or biologic therapy, or any combination thereof.

In another embodiment, the invention includes a method for diagnosing and treating a patient suspected or at risk of having breast cancer, the method comprising: a) contacting breast tissue of the patient with a detectably effective amount of a B7-H3-targeted imaging agent under conditions wherein the B7-H3-targeted imaging agent binds to B7-H3 on any breast tumor neovasculature, breast tumor epithelium, cancerous cells, or precancerous lesions, if present, in the breast tissue; b) diagnosing the patient by imaging breast tissue of the patient, wherein detection of increased binding of the B7-H3-targeted imaging agent to the breast tissue of the patient compared to a control indicates that the patient has breast cancer; and c) administering anti-cancer therapy to the subject if the patient is diagnosed with breast cancer. In certain embodiments, the anti-cancer therapy comprises surgery, radiation therapy, chemotherapy, hormonal therapy, immunotherapy, or biologic therapy, or any combination thereof.

In certain embodiments, the anti-cancer treatment comprises surgical removal of at least a portion of a breast, for example, by performing a mastectomy (removal of the whole breast), quadrantectomy (removal of one quarter of the breast), or lumpectomy (removal of a small part of the breast).

In another embodiment, a subject diagnosed with breast cancer by a method described herein may be administered a therapeutically effective amount of a hormonal blocking therapeutic agent selected from the group consisting of tamoxifen, anastrozole, and letrozole.

In another embodiment, a subject diagnosed with breast cancer by a method described herein may be administered a therapeutically effective amount of a chemotherapeutic agent selected from the group consisting of cyclophosphamide, doxorubicin, docetaxel, cyclophosphamide, methotrexate, and fluorouracil.

In another embodiment, a subject diagnosed with breast cancer by a method described herein may be administered a therapeutically effective amount of an anti-HER2 receptor antibody (e.g., Trastuzumab).

These and other embodiments of the subject invention will readily occur to those of skill in the art in view of the disclosure herein.

### BRIEF DESCRIPTION OF THE DRAWINGS

FIGS. 1A-1C show a summary of the overall study design. FIG. 1A shows differential expression of B7-H3 on breast cancer-associated neovasculature assessed on a panel of normal, benign, premalignant, and malignant breast lesions obtained from women undergoing biopsy or surgical resection. FIG. 1B shows development of B7-H3-targeted contrast microbubbles. FIG. 1C shows testing of the B7-H3-targeted contrast microbubbles both in cell culture and in vivo in a transgenic mouse model of breast cancer.

FIGS. 2A-2D show immunohistochemistry (IHC) analysis of B7-H3 expression in human breast tissues, FIGS. 2A-2C show photomicrographs representative of staining results from normal breast tissues, various benign, and precursor breast pathologies, as well as different types of breast cancer obtained from women undergoing biopsy or surgical resection. FIG. 2A shows atypical ductal hyperplasia (ADH); atypical lobular hyperplasia (ALE); apocrine metaplasia (ApoM); columnar cell lesion (CCL); ductal carcinoma in situ (DCIS); FIG. 2B shows fibroadenoma (FA); Flat epithelial atypia (FEA); nonproliferative fibrocystic changes (NPFCC); usual ductal hyperplasia (UDH); FIG. 2C shows Luminal A, estrogen receptor and/or progesterone receptor-positive cancer, Luminal B, estrogen receptor and/or progesterone receptor-positive and Her2-positive cancer, and triple negative, estrogen, progesterone, and Her2-negative cancer. FIG. 2D shows a graph summarizing composite IHC scores on B7-H3-stained tissues from normal tissue, benign and precursor lesions versus breast cancer. *, P<0.001; error bars, SD; scale bar, 100 μm.

FIG. 3 shows a summary of composite IHC scores of B7-H3 staining of the vasculature in normal breast tissue, benign, premalignant, and malignant breast lesions. *, P<0.001; error bars, SD.

FIG. 4 shows a microvessel density (MVD) analysis on CD31-stained normal breast tissue, benign, premalignant, and malignant lesions. *, P<0.001; error bars, SD.

FIG. 5 shows in vitro binding specificity of B7-H3-targeted microbubbles. Representative photomicrographs from cell culture experiments using a parallel plate flow chamber setting with B7-H3-positive and B7-H3-negative vascular endothelial cells exposed to B7-H3-targeted microbubbles (of MB_{B7-H3}) and nontargeted control microbubbles (of MB_{Control}). Note specific attachment of MB_{B7-H3} to B7-H3-positive cells and substantial binding inhibition following administration of blocking antibodies. Microbubbles (arrows) are visualized as white spherical dots. *, P<0.01; error bars, SD.

FIGS. 6A-6D show in vivo ultrasound molecular imaging. FIG. 6A shows a representative transverse B-mode and contrast mode ultrasound images following injection of B7-H3-targeted contrast microbubbles showing a strong signal in breast cancer and only background signal in a mammary gland with normal breast tissue (both outlined by a gray triangular region of interest). FIG. 6B shows photomicrographs of immunofluorescence images [double stained for both the vascular marker CD31 and B7-H3] confirm expression of B7-H3 on tumor neovasculature (arrows, signal on merged images) in breast cancer with little to no vascular expression in normal tissue. Note B7-H3 is also expressed on tumor epithelium (arrowheads). FIG. 6C shows a bar graph summarizes quantitative B7-H3-targeted ultrasound molecular imaging signal obtained in normal and breast cancer in a total of 183 mammary glands, with significantly increased imaging signal in breast cancer versus normal tissue. *, P<0.001; error bars, SD. FIG. 6D shows a ROC curve distinguishing normal from breast cancer based on quantitative ultrasound molecular imaging signal.

FIG. 7 shows a summary of composite immunohistochemical vascular staining scores of B7-H3 staining in whole tissue breast cancer samples and surrounding normal, benign and precursor breast lesions. \hat{ }P<0.01; *P<0.001; error bars=standard deviations.

FIG. 8 shows a summary of microvessel density analyzed in whole tissue breast cancer samples and surrounding normal, benign and precursor breast lesions. *P<0.001; error bars=standard deviations

FIG. 9 shows binding specificity of MB_{B7-H3}. A summary is shown of ultrasound molecular imaging signals obtained after intravenous administration of MB_{B7-H3}, MB_{Control} and MB_{B7-H3} after blocking with anti-B7-H3 antibodies (*P<0.001) in the same animal in a given imaging session. Representative transverse B-mode and contrast mode ultrasound images obtained after administration of MB_{B7-H3} and MB_{Control} are shown. The region of interest (ROI), (mammary gland) is outlined with a gray triangle.

FIGS. 10A and 10B show an evaluation of B7-H3 expression on vascular endothelial cells. FIG. 10A shows the expression levels of B7-H3 in MS1-wild-type (wt) and MS1-B7-H3 cells as quantitatively assessed by fluorescence-activated cell sorting analysis and histogram overlays of signals from these cells. FIG. 10B shows bar graphs of mean fluorescence intensity values (mean±SD).

### DETAILED DESCRIPTION

The practice of the present invention will employ, unless otherwise indicated, conventional methods of medicine, pharmacology, chemistry, biochemistry, recombinant DNA techniques and immunology, within the skill of the art. Such techniques are explained fully in the literature. See, e.g., D. B. Kopans Breast Imaging (Kopans, Breast Imaging, LWW, Third edition, 2006); T. Stavros Breast Ultrasound (LWW, 2003); M. Tartar, C. E. Comstock, M. S. Kipper Breast Cancer Imaging: A Multidisciplinary, Multimodality Approach, 1e (Mosby, 2008); A. B. Wolbarst et al. Medical Imaging: Essentials for Physicians (Wiley-Blackwell, 2013); Handbook of Experimental Immunology, Vols. I-IV (D. M. Weir and C. C. Blackwell eds., Blackwell Scientific Publications); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.).

All publications, patents and patent applications cited herein, whether supra or infra, are hereby incorporated by reference in their entireties.

### I. Definitions

In describing the present invention, the following terms will be employed, and are intended to be defined as indicated below.

It must be noted that, as used in this specification and the appended claims, the singular forms “a”, “an” and “the” include plural referents unless the content clearly dictates otherwise. Thus, for example, reference to “an antibody” includes a mixture of two or more antibodies, and the like.

The term “about”, particularly in reference to a given quantity, is meant to encompass deviations of plus or minus five percent.

The terms “tumor,” “cancer” and “neoplasia” are used interchangeably and refer to a cell or population of cells whose growth, proliferation or survival is greater than growth, proliferation or survival of a normal counterpart cell, e.g. a cell proliferative, hyperproliferative or differentiative disorder. Typically, the growth is uncontrolled. The term “malignancy” refers to invasion of nearby tissue. The term “metastasis” or a secondary, recurring or recurrent tumor, cancer or neoplasia refers to spread or dissemination of a tumor, cancer or neoplasia to other sites, locations or regions within the subject, in which the sites, locations or regions are distinct from the primary tumor or cancer. Neoplasia, tumors and cancers include benign, malignant, metastatic and non-metastatic types, and include any stage (I, II, II, IV or V) or grade (G1, G2, G3, etc.) of neoplasia, tumor, or cancer, or a neoplasia, tumor, cancer or metastasis that is progressing, worsening, stabilized or in remission

The terms “subject,” “individual,” and “patient,” are used interchangeably herein and refer to any mammalian subject for whom diagnosis, prognosis, treatment, or therapy is desired, particularly humans. Other subjects may include cattle, dogs, cats, guinea pigs, rabbits, rats, mice, horses, and so on. In some cases, the methods of the invention find use in experimental animals, in veterinary application, and in the development of animal models for disease, including, but not limited to, rodents including mice, rats, and hamsters; and primates.

The terms “quantity,” “amount,” and “level” are used interchangeably herein and may refer to an absolute quantification of a molecule or an analyte (e.g., B7-H3), or to a relative quantification of a molecule or analyte, i.e., relative to another value such as relative to a reference value as taught herein, or to a range of values for the molecule or analyte. These values or ranges can be obtained from a single patient or from a group of patients.

The term “antibody” encompasses polyclonal and monoclonal antibody preparations, as well as preparations including hybrid antibodies, altered antibodies, chimeric antibodies and, humanized antibodies, as well as: hybrid (chimeric) antibody molecules (see, for example, Winter et al. (1991) Nature 349:293-299; and U.S. Pat. No. 4,816,567); F(ab′)₂ and F(ab) fragments; Fᵥ molecules (noncovalent heterodimers, see, for example, Inbar et al. (1972) Proc Natl Acad Sci USA 69:2659-2662; and Ehrlich et al. (1980) Biochem 19:4091-4096); single-chain Fv molecules (sFv) (see, e.g., Huston et al. (1988) Proc Natl Acad Sci USA 85:5879-5883); dimeric and trimeric antibody fragment constructs; minibodies (see, e.g., Pack et al. (1992) Biochem 31:1579-1584; Cumber et al. (1992) J Immunology 149B:120-126); humanized antibody molecules (see, e.g., Riechmann et al. (1988) Nature 332:323-327; Verhoeyan et al. (1988) Science 239:1534-1536; and U.K. Patent Publication No. GB 2,276,169, published 21 Sep. 1994); and, any functional fragments obtained from such molecules, wherein such fragments retain specific-binding properties of the parent antibody molecule.

A “single-chain antibody,” “single chain variable fragment,” or “scFv” comprises an antibody heavy chain variable domain (VH) and a light-chain variable domain (VL) joined together by a flexible peptide linker. The peptide linker is typically 10-25 amino acids in length. Single-chain antibodies retain the antigen-binding properties of natural full-length antibodies, but are smaller than natural intact antibodies or Fab fragments because of the lack of an Fc domain.

“Immunoassay” is an assay that uses an antibody to specifically bind an antigen (e.g., B7-H3). The immunoassay is characterized by the use of specific binding properties of a particular antibody to isolate, target, and/or quantify the antigen. An immunoassay for detection of an antigen may utilize one antibody or several antibodies. Immunoassay protocols may be based, for example, upon competition, direct reaction, or sandwich type assays using, for example, a labeled antibody. The labels may be, for example, fluorescent, chemiluminescent, or radioactive. Alternatively, the antibody may be conjugated to a diagnostic agent, such as a contrast agent or photoactive agent that is useful for biomedical imaging (e.g., ultrasound, MRI, or CT)

The phrase “specifically (or selectively) binds” when referring to binding of a B7-H3 targeting agent to B7-H3 (or a peptide fragment thereof), refers to a binding reaction that is determinative of the presence of B7-H3 in a heterogeneous population of proteins and other biologics. Thus, under designated conditions, the specified B7-H3 targeting agents bind to B7-H3 at least two times the background and do not substantially bind in a significant amount to other proteins present. Specific binding of a B7-H3 targeting agent under such conditions may require that the targeting agent is selected for its specificity for B7-H3. Typically specific or selective binding will be at least twice the background signal or noise and more typically more than 10 to 100 times background.

An antibody binds “essentially the same epitope” as a reference antibody, when the two antibodies recognize identical or sterically overlapping epitopes. The most widely used and rapid methods for determining whether two antibodies bind to identical or sterically overlapping epitopes are competition assays, which can be configured in all number of different formats, using either labeled antigen or labeled antibody. Usually, the antigen is immobilized on a substrate, and the ability of unlabeled antibodies to block the binding of labeled antibodies is measured using radioactive isotopes or enzyme labels.

The term “B7-H3-targeting ligand” or “B7-H3-targeting agent” refers to any molecule that selectively binds to B7-H3 that is capable of localizing an imaging or detection agent to B7-H3 on the surface of cancerous cells or tissue (e.g. breast tumor neovasculature or epithelium). B7-H3-targeting agents may include antibodies, peptides, peptoids, aptamers, small molecule ligands or any other agent that selectively binds to B7-H3.

A “B7-H3-targeted diagnostic agent” or “B7-H3-targeted imaging agent” refers to a B7-H3-targeting agent that is detectably labeled or conjugated to a diagnostic or detection agent. Such B7-H3-targeted diagnostic or imaging agents can be used in detection, diagnosis, or medical imaging of breast cancer.

As used herein, the terms “detection agent”, “diagnostic agent”, and “detectable label” refer to a molecule or substance capable of detection, including, but not limited to, fluorescers, chemiluminescers, chromophores, bioluminescent proteins, enzymes, enzyme substrates, enzyme cofactors, enzyme inhibitors, isotopic labels, semiconductor nanoparticles, dyes, metal ions, metal sols, ligands (e.g., biotin, streptavidin or haptens) and the like. The term “fluorescer” refers to a substance or a portion thereof which is capable of exhibiting fluorescence in the detectable range. Particular examples of labels which may be used in the practice of the invention include, but are not limited to, SYBR green, SYBR gold, a CAL Fluor dye such as CAL Fluor Gold 540, CAL Fluor Orange 560, CAL Fluor Red 590, CAL Fluor Red 610, and CAL Fluor Red 635, a Quasar dye such as Quasar 570, Quasar 670, and Quasar 705, an Alexa Fluor such as Alexa Fluor 350, Alexa Fluor 488, Alexa Fluor 546, Alexa Fluor 555, Alexa Fluor 594, Alexa Fluor 647, and Alexa Fluor 784, a cyanine dye such as Cy 3, Cy3.5, Cy5, Cy5.5, and Cy7, fluorescein, 2′,4′, 5′,7′-tetrachloro-4-7-dichlorofluorescein (TET), carboxyfluorescein (FAM), 6-carboxy-4′,5′-dichloro-2′,7′-dimethoxyfluorescein (JOE), hexachlorofluorescein (HEX), rhodamine, carboxy-X-rhodamine (ROX), tetramethyl rhodamine (TAMRA), FITC, dansyl, umbelliferone, dimethyl acridinium ester (DMAE), Texas red, luminol, and quantum dots, enzymes such as alkaline phosphatase (AP), beta-lactamase, chloramphenicol acetyltransferase (CAT), adenosine deaminase (ADA), aminoglycoside phosphotransferase (neor, G418r) dihydrofolate reductase (DHFR), hygromycin-B-phosphotransferase (HPH), thymidine kinase (TK), β-galactosidase (lacZ), and xanthine guanine phosphoribosyltransferase (XGPRT), beta-glucuronidase (gus), placental alkaline phosphatase (PLAP), and secreted embryonic alkaline phosphatase (SEAP). Enzyme tags are used with their cognate substrate. The terms also include chemiluminescent labels such as luminol, isoluminol, acridinium esters, and peroxyoxalate and bioluminescent proteins such as firefly luciferase, bacterial luciferase, Renilla luciferase, and aequorin. The terms also include isotopic labels, including radioactive and non-radioactive isotopes, such as, ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr. The terms also include color-coded microspheres of known fluorescent light intensities (see e.g., microspheres with xMAP technology produced by Luminex (Austin, Tex.); microspheres containing quantum dot nanocrystals, for example, containing different ratios and combinations of quantum dot colors (e.g., Qdot nanocrystals produced by Life Technologies (Carlsbad, Calif.); glass coated metal nanoparticles (see e.g., SERS nanotags produced by Nanoplex Technologies, Inc. (Mountain View, Calif.); barcode materials (see e.g., sub-micron sized striped metallic rods such as Nanobarcodes produced by Nanoplex Technologies, Inc.), encoded microparticles with colored bar codes (see e.g., CellCard produced by Vitra Bioscience, vitrabio.com), glass microparticles with digital holographic code images (see e.g., CyVera microbeads produced by Illumina (San Diego, Calif.), near infrared (NIR) probes, and nanoshells. The terms also include contrast agents such as ultrasound contrast agents (e.g. SonoVue microbubbles comprising sulfur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate).

As used herein, a “microbubble” refers to a micron-sized contrast agent composed of a shell and a gas core. The shell may be formed from any suitable material, including, but not limited to, proteins (e.g., albumin), polysaccharides (e.g., galactose), lipids (such as phospholipids), polymers, and combinations thereof. Any suitable gas core can be used in microbubbles, including, but not limited to, air, octafluoropropane, perfluorocarbon, sulfur hexafluoride, or nitrogen. Microbubbles can be used, for example, as contrast agents for ultrasound imaging. The microbubbles oscillate and vibrate when a sonic energy field is applied and reflect ultrasound waves. The gas core determines the echogenecity of the microbubble. The average diameter of a microbubble is typically between about 1 μm and about 25 μm. In general, microbubbles have a diameter ranging between about 1 μm and about 10 μm on average, and more preferably between about 1 μm and 5 μm, 1 μm and 4 μm, 1 μm and 3 μm, 1 μm and about 2 μm, 2 μm and 5 μm, 2 μm and 4 μm, 2 μm and 3 μm, 3 μm and 5 μm, 3 μm and 4 μm, or about 1 μm, 2 μm, 2.5 μm, 3 μm, 3.5 μm, or 4 μm on average.

Following administration to at patient (such as by intravenous injection), B7-H3-targeted microbubbles (e.g., conjugated to an anti-B7-H3 antibody) accumulate at tissue sites that over-express B7-H3 causing a local increase in the ultrasound imaging signal. Microbubbles can be used, for example, to detect B7-H3 antigen on the surface of breast tumor neovasculature or precancerous lesions that are present in early stage breast cancer.

“Diagnosis” as used herein generally includes determination as to whether a subject is likely affected by a given disease, disorder or dysfunction. The skilled artisan often makes a diagnosis on the basis of one or more diagnostic indicators, i.e., a biomarker, the presence, absence, or amount of which is indicative of the presence or absence of the disease, disorder or dysfunction.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

The terms “polypeptide” and “protein” refer to a polymer of amino acid residues and are not limited to a minimum length. Thus, peptides, oligopeptides, dimers, multimers, and the like, are included within the definition. Both full length proteins and fragments thereof are encompassed by the definition. The terms also include postexpression modifications of the polypeptide, for example, glycosylation, acetylation, phosphorylation, hydroxylation, and the like. Furthermore, for purposes of the present invention, a “polypeptide” refers to a protein which includes modifications, such as deletions, additions and substitutions to the native sequence, so long as the protein maintains the desired activity. These modifications may be deliberate, as through site directed mutagenesis, or may be accidental, such as through mutations of hosts which produce the proteins or errors due to PCR amplification.

By “derivative” is intended any suitable modification of the native polypeptide of interest, of a fragment of the native polypeptide, or of their respective analogs, such as glycosylation, phosphorylation, polymer conjugation (such as with polyethylene glycol), or other addition of foreign moieties, as long as the desired biological activity of the native polypeptide is retained. Methods for making polypeptide fragments, analogs, and derivatives are generally available in the art.

By “fragment” is intended a molecule consisting of only a part of the intact full length sequence and structure. The fragment can include a C-terminal deletion an N-terminal deletion, and/or an internal deletion of the polypeptide. Active fragments of a particular protein or polypeptide will generally include at least about 5-10 contiguous amino acid residues of the full length molecule, preferably at least about 15-25 contiguous amino acid residues of the full length molecule, and most preferably at least about 20-50 or more contiguous amino acid residues of the full length molecule, or any integer between 5 amino acids and the full length sequence, provided that the fragment in question retains biological activity, such as catalytic activity or ligand binding activity.

“Substantially purified” generally refers to isolation of a substance (compound, polynucleotide, antibody, protein, polypeptide, polypeptide composition) such that the substance comprises the majority percent of the sample in which it resides. Typically in a sample, a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

By “isolated” is meant, when referring to a polypeptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

The term “transformation” refers to the insertion of an exogenous polynucleotide into a host cell, irrespective of the method used for the insertion. For example, direct uptake, transduction or f-mating are included. The exogenous polynucleotide may be maintained as a non-integrated vector, for example, a plasmid, or alternatively, may be integrated into the host genome.

By “anti-tumor activity” is intended a reduction in the rate of cell proliferation, and hence a decline in growth rate of an existing tumor or in a tumor that arises during therapy, and/or destruction of existing neoplastic (tumor) cells or newly formed neoplastic cells, and hence a decrease in the overall size of a tumor during therapy. Such activity can be assessed using animal models.

The term “survival” as used herein means the time from the first dose of a B7-H3-targeted therapeutic agent (e.g., a bioconjugate comprising an anti-B7-H3 antibody conjugated to an anti-cancer therapeutic agent) to the time of death.

By “therapeutically effective dose or amount” of a B7-H3-targeted therapeutic agent (e.g., a bioconjugate comprising an anti-B7-H3 antibody conjugated to an anti-cancer therapeutic agent) is intended an amount that, when administered as described herein, brings about a positive therapeutic response, such as an amount that has anti-tumor activity, inhibits metastasis, or increases survival of a subject treated for a breast cancer. The exact amount required will vary from subject to subject, depending on the species, age, and general condition of the subject, the severity of the condition being treated, the particular drug or drugs employed, mode of administration, and the like. An appropriate “effective” amount in any individual case may be determined by one of ordinary skill in the art using routine experimentation, based upon the information provided herein.

The term “tumor response” as used herein means a reduction or elimination of all measurable lesions. The criteria for tumor response are based on the WHO Reporting Criteria [WHO Offset Publication, 48-World Health Organization, Geneva, Switzerland, (1979)]. Ideally, all uni- or bidimensionally measurable lesions should be measured at each assessment. When multiple lesions are present in any organ, such measurements may not be possible and, under such circumstances, up to 6 representative lesions should be selected, if available.

The term “complete response” (CR) as used herein means a complete disappearance of all clinically detectable malignant disease, determined by 2 assessments at least 4 weeks apart.

The term “partial response” (PR) as used herein means a 50% or greater reduction from baseline in the sum of the products of the longest perpendicular diameters of all measurable disease without progression of evaluable disease and without evidence of any new lesions as determined by at least two consecutive assessments at least four weeks apart. Assessments should show a partial decrease in the size of lytic lesions, recalcifications of lytic lesions, or decreased density of blastic lesions.

### II. Modes of Carrying Out the Invention

Before describing the present invention in detail, it is to be understood that this invention is not limited to particular formulations or process parameters as such may, of course, vary. It is also to be understood that the terminology used herein is for the purpose of describing particular embodiments of the invention only, and is not intended to be limiting.

Although a number of methods and materials similar or equivalent to those described herein can be used in the practice of the present invention, the preferred materials and methods are described herein.

The present invention is based on the discovery that vascular expression of B7-H3 (also known as CD276) can be used to differentiate normal, benign, precancerous, and malignant breast pathologies for diagnostic purposes (Example 1). The B7-H3 antigen, which is overexpressed on breast tumor neovasculature and epithelium, and to a lesser extent on precancerous lesions, can be detected using B7-H3-targeted imaging agents. The inventors produced B7-H3-targeted contrast agents for ultrasound molecular imaging by conjugating anti-B7-H3 antibodies to microbubbles. The inventors further showed that such targeted microbubbles were effective in detection and diagnosis of breast cancer in patients (Example 1). Anti-B7-H3 antibodies may also be conjugated to other diagnostic agents, including other types of contrast agents, photoactive agents, or detectable labels for use in medical imaging and detection of breast cancer. In addition, anti-B7-H3 antibodies may be conjugated to therapeutic agents for targeted treatment of breast cancer.

In order to further an understanding of the invention, a more detailed discussion is provided below regarding B7-H3-targeted imaging agents and their use in detection and diagnosis of breast cancer as well as medical imaging and therapeutics.

A. B7-H3 Targeting of Breast Cancer

The methods of the invention utilize B7-H3-targeted imaging agents that selectively bind to breast tumor neovasculature, breast tumor epithelium, and precancerous lesions, which overexpress the B7-H3 antigen. Such B7-H3-targeted imaging agents selectively bind to cancerous breast tissue and not to normal or benign breast tissue and can be used for diagnosing breast cancer as well as differentiating breast cancer from benign and precursor breast lesions. In particular, B7-H3-targeted imaging agents can be used to distinguish breast cancer from other non-cancerous conditions such as adenosis, atypical ductal hyperplasia (ADH), atypical lobular hyperplasia (ALH), apocrine metaplasia (ApoM), columnar cell lesion (CCL), ductal carcinoma in situ (DCIS), fibroadenoma (FA), flat epithelial atypia (FEA), nonproliferative fibrocystic changes (NPFCC), radial scar, and usual ductal hyperplasia (UDH). Moreover, B7-H3-targeted imaging agents are useful for detecting and diagnosing various types of breast cancer, including, but not limited to, luminal A breast cancer, luminal B breast cancer, triple negative breast cancer, and Her2-positive breast cancer.

The B7-H3-targeted imaging agents comprise a diagnostic agent conjugated to a targeting agent that selectively binds to B7-H3. B7-H3-targeting agents may include antibodies, peptides, peptoids, aptamers, small molecule ligands, or any other agents that bind selectively to B7-H3. Such B7-H3-targeting agents are capable of localizing an imaging or detection agent to B7-H3 on the surface of cancerous cells or tissue, including breast tumor neovasculature or epithelium.

In one embodiment, the B7-H3-targeting agent is an antibody. Antibodies that specifically bind to the B7-H3 antigen can be prepared using any suitable methods known in the art. See, e.g., Coligan, Current Protocols in Immunology (1991); Harlow & Lane, Antibodies: A Laboratory Manual (1988); Goding, Monoclonal Antibodies: Principles and Practice (2d ed. 1986); and Kohler & Milstein, Nature 256:495-497 (1975). B7-H3 can be used to immunize a mammal, such as a mouse, rat, rabbit, guinea pig, monkey, or human, to produce polyclonal antibodies. If desired, the B7-H3 antigen can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g. lysolecithin, pluronic polyols, polyanions, peptides, oil emulsions, keyhole limpet hemocyanin, and dinitrophenol). Among adjuvants used in humans, BCG (bacilli Calmette-Guerin) and Corynebacterium parvum are especially useful.

Monoclonal antibodies which specifically bind to the B7-H3 antigen can be prepared using any technique which provides for the production of antibody molecules by continuous cell lines in culture. These techniques include, but are not limited to, the hybridoma technique, the human B cell hybridoma technique, and the EBV hybridoma technique (Kohler et al., Nature 256, 495-97, 1985; Kozbor et al., J. Immunol. Methods 81, 31 42, 1985; Cote et al., Proc. Natl. Acad. Sci. 80, 2026-30, 1983; Cole et al., Mol. Cell Biol. 62, 109-20, 1984).

In addition, techniques developed for the production of “chimeric antibodies,” such as the splicing of antibody genes from a mouse (or other species) to human antibody genes to obtain a molecule with appropriate antigen specificity and biological activity, can be used (Morrison et al., Proc. Natl. Acad. Sci. 81, 6851-55, 1984; Neuberger et al., Nature 312, 604-08, 1984; Takeda et al., Nature 314, 452-54, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site directed mutagenesis of individual residues or by grafting of entire complementarity determining regions.

Alternatively, humanized antibodies can be produced using recombinant methods, as described below. Antibodies which specifically bind to a particular antigen can contain antigen binding sites which are either partially or fully humanized, as disclosed in U.S. Pat. No. 5,565,332. Human monoclonal antibodies can be prepared in vitro as described in Simmons et al., PLoS Medicine 4(5), 928-36, 2007.

Alternatively, techniques described for the production of single-chain antibodies can be adapted using methods known in the art to produce single-chain antibodies that specifically bind to the B7-H3 antigen. Single-chain antibodies comprise an antibody heavy chain variable domain (VH) and a light-chain variable domain (VL) joined together by a flexible peptide linker (e.g., typically 10-25 amino acids in length). Advantages of using single-chain antibodies include that they retain the antigen-binding properties of natural full-length antibodies, but lacking the Fc domain, are smaller, have better tumor penetration, and do not stimulate Fc-mediated immune effector functions.

Antibodies with related specificity, but of distinct idiotypic composition, can be generated by chain shuffling from random combinatorial immunoglobulin libraries (Burton, Proc. Natl. Acad. Sci. 88, 11120-23, 1991). Single-chain antibodies may also be constructed using a DNA amplification method, such as PCR, using hybridoma cDNA as a template (Thirion et al., Eur. J. Cancer Prev. 5, 507-11, 1996). A nucleotide sequence encoding a single-chain antibody can be constructed using manual or automated nucleotide synthesis, cloned into an expression construct using standard recombinant DNA methods, and introduced into a cell to express the coding sequence, as described below. Alternatively, single-chain antibodies can be produced directly using, for example, filamentous phage technology (Verhaar et al., Int. J. Cancer 61, 497-501, 1995; Nicholls et al., J. Immunol. Meth. 165, 81-91, 1993).

Antibodies which specifically bind to the B7-H3 antigen also can be produced by inducing in vivo production in the lymphocyte population or by screening immunoglobulin libraries or panels of highly specific binding reagents as disclosed in the literature (Orlandi et al., Proc. Natl. Acad. Sci. 86, 3833 3837, 1989; Winter et al., Nature 349, 293 299, 1991).

Chimeric antibodies can be constructed as disclosed in WO 93/03151. Binding proteins which are derived from immunoglobulins and which are multivalent and multispecific, such as the “diabodies” described in WO 94/13804, also can be prepared.

Anti-B7-H3 antibodies can be purified by methods well known in the art. For example, such antibodies can be affinity purified by passage over a column to which the B7-H3 antigen is bound. The bound antibodies can then be eluted from the column using a buffer with a high salt concentration.

In another embodiment, the B7-H3-targeting agent is an aptamer. The aptamer may be an oligonucleotide or peptide that selectively binds to B7-H3 identified, for example, by screening a combinatorial library. Nucleic acid aptamers (e.g., DNA or RNA aptamers) that bind selectively to B7-H3 can be produced by carrying out repeated rounds of in vitro selection or systematic evolution of ligands by exponential enrichment (SELEX). Peptide aptamers that bind to B7-H3 may be isolated from a combinatorial library and improved by directed mutation or repeated rounds of mutagenesis and selection. For a description of methods of producing aptamers, see, e.g., Aptamers: Tools for Nanotherapy and Molecular Imaging (R. N. Veedu ed., Pan Stanford, 2016), Nucleic Acid and Peptide Aptamers: Methods and Protocols (Methods in Molecular Biology, G. Mayer ed., Humana Press, 2009), Nucleic Acid Aptamers: Selection, Characterization, and Application (Methods in Molecular Biology, G. Mayer ed., Humana Press, 2016), Aptamers Selected by Cell-SELEXfor Theranostics (W. Tan, X. Fang eds., Springer, 2015), Cox et al. (2001) Bioorg. Med. Chem. 9(10):2525-2531; Cox et al. (2002) Nucleic Acids Res. 30(20): e108, Kenan et al. (1999) Methods Mol Biol. 118:217-231; Platella et al. (2016) Biochim. Biophys. Acta November 16 pii: S0304-4165(16)30447-0, and Lyu et al. (2016) Theranostics 6(9):1440-1452; herein incorporated by reference in their entireties.

The methods described herein for detection and diagnosis of breast cancer with B7-H3-targeted imaging agents may be used in individuals who have not yet been diagnosed (for example, preventative screening), or who have been diagnosed, or who are suspected or at risk of having breast cancer (e.g., display one or more characteristic symptoms, presence of breast tumors), or who are at risk of developing breast cancer (e.g., have precancerous lesions). The methods may also be used to detect various stages of progression or severity of disease (e.g., benign, premalignant, and malignant breast lesions, tumor growth, or metastasis). The methods may also be used to detect the response of the disease to prophylactic or therapeutic treatments or other interventions. The methods can furthermore be used to help the medical practitioner in determining prognosis (e.g., worsening, status-quo, partial recovery, or complete recovery) of the patient, and the appropriate course of action, resulting in either further treatment or observation, or in discharge of the patient from the medical care center.

Anti-cancer therapy may be administered to a patient found to have a positive diagnosis for breast cancer based on detection of overexpression of B7-H3 on breast cancer neovasculature or epithelium, as described herein. Anti-cancer therapy may comprise one or more of surgery, radiation therapy, chemotherapy, hormonal therapy, immunotherapy, or biologic therapy. For example, breast cancer may be treated by surgical removal of at least a portion of a breast, for example, by performing a mastectomy (removal of the whole breast), quadrantectomy (removal of one quarter of the breast), or lumpectomy (removal of a small part of the breast). Alternatively or additionally, a patient diagnosed with breast cancer may be administered monoclonal antibody therapy with an anti-HER2 receptor antibody (e.g., Trastuzumab), chemotherapy with one or more chemotherapeutic agents (e.g., cyclophosphamide, doxorubicin, docetaxel, cyclophosphamide, methotrexate, and fluorouracil), or hormonal blocking therapy with a drug that blocks estrogen receptors (e.g. tamoxifen) or a drug that blocks the production of estrogen, such as an aromatase inhibitor (e.g. anastrozole or letrozole); or any combination thereof.

B. Bioconjugation

Bioconjugates may comprise one or more diagnostic or therapeutic agents, or a combination thereof, conjugated to a B7-H3-targeting agent. The B7-H3-targeting agent may be attached to the diagnostic and/or therapeutic agents in a variety of manners. For example, an agent may be attached at the N-terminus, C-terminus, at both the N-terminus and C-terminus, and/or internally. Diagnostic and/or therapeutic agents may be connected directly to the B7-H3-targeting agent or indirectly through an intervening linker or chelating agent (e.g., for metal labeling such as with a radionuclide or paramagnetic metal ion).

Bioconjugation can be performed using methods well-known in the art. For a discussion of bioconjugation techniques, see, e.g., Chemistry of Bioconjugates: Synthesis, Characterization, and Biomedical Applications (R. Narain ed., Wiley, 2014), G. T. Hermanson Bioconjugate Techniques (Academic Press, 3^{rd} edition, 2013), and Bioconjugation Protocols: Strategies and Methods (Methods in Molecular Biology, S. S. Mark ed., Humana Press, 2^{nd} edition, 2011), van Vught et al. (2014) Comput Struct Biotechnol J. 9:e201402001; Gao et al. (2016) Curr Cancer Drug Targets 16(6):469-479; Massa et al. (2016) Expert Opin. Drug Deliv. 13:1-15; Yeh et al. (2015) PLoS One 10(7):e0129681; Freise et al. (2015) Mol Immunol. 67(2 Pt A): 142-152; herein incorporated by reference in their entireties.

For example, imaging and/or therapeutic agents can be conjugated to the side chain ε-amine of lysine residues or the free thiol of cysteine residues. In particular, reactions of cysteine thiols with maleimides are commonly used for bioconjugation of proteins. Maleimide-functionalized imaging probes and compounds to facilitate bioconjugation for various imaging modalities are commercially available from a number of companies (e.g., ThermoFisher Scientific (Waltham, Mass.), GE Healthcare Life Sciences (Pittsburgh, Pa.), SigmaAldrich (St. Louis, Mo.), and CHEMATECH (Dijon, France)), including, for example, maleimide lipid derivatives and maleimide albumin derivatives, which can be incorporated into a microbubble shell for ultrasound imaging, maleimide fluorescent dye derivatives for fluorescence imaging, maleimide chelating agent derivatives for binding metals such as radionuclides and paramagnetic cations, and maleimide gold nanoparticle derivatives, which can be used in a variety of ways including as detection agents for electron microscopy and surface enhanced Raman spectroscopy, enhancement agents for radiotherapy, photothermal agents for surface plasmon resonance, and delivery agents for attached drugs or other therapeutic agents.

In certain embodiments, B7-H3-targeting agent is engineered to include an N-terminal or C-terminal cysteine residue providing a free thiol group to facilitate conjugation to a reagent comprising a functional group that is reactive with thiols. In other embodiments, a cysteine is incorporated into a linker peptide to allow attachment of reagents to a linker connected to the B7-H3-targeting agent. Alternatively, additional cysteine residues may be introduced into the B7-H3-targeting agent, for example, by site-directed mutagenesis to allow attachment at other sites. A site of attachment away from the B7-H3 binding site should be chosen to avoid interfering with B7-H3 targeting of the bioconjugate.

An alternative bioconjugation method uses click chemistry. Click chemistry reactions include the Huisgen 1,3-dipolar cycloaddition copper catalyzed reaction (Tornoe et al., 2002, J Organic Chem 67:3057-64), cycloaddition reactions such as Diels-Alder reactions, nucleophilic substitution reactions (especially to small strained rings like epoxy and aziridine compounds), reactions involving formation of urea compounds, and reactions involving carbon-carbon double bonds, such as alkynes in thiol-yne reactions. See, e.g., Kolb et al., 2004, Angew Chem Int Ed 40:3004-31; Evans, 2007, Aust J Chem 60:384-95; Millward et al. (2013) Integr Biol (Camb) 5(1):87-95), Lallana et al. (2012) Pharm Res 29(1):1-34, Gregoritza et al. (2015) Eur J Pharm Biopharm. 97(Pt B):438-453, Musumeci et al. (2015) Curr Med Chem. 22(17):2022-2050, McKay et al. (2014) Chem Biol21(9):1075-1101, Ulrich et al. (2014) Chemistry 20(1):34-41, Pasini (2013) Molecules 18(8):9512-9530, and Wangler et al. (2010) Curr Med Chem. 17(11): 1092-1116; herein incorporated by reference in their entireties.

B7-H3-Targeted Imaging Agents

B7-H3-targeting agents can be conjugated to diagnostic agents (e.g., probes or detection agents) suitable for various imaging modalities, including, but not limited to, ultrasound imaging (UI), positron emission tomography (PET), single photon emission computed tomography (SPECT), magnetic resonance imaging (MRI), computed tomography (CT), optical imaging (OI), photoacoustic imaging (PI), fluoroscopy, or fluorescence imaging. Conjugation of a diagnostic agent comprising a detectable moiety or label to a B7-H3-targeting agent localizes the diagnostic agent to cancerous or pre-cancerous cells expressing the B7-H3 antigen. Useful diagnostic agents that can be used in the practice of the invention include, but are not limited to, contrast agents, photoactive agents, radioisotopes, nonradioactive isotopes, dyes, fluorescent compounds or proteins, chemiluminescent compounds, bioluminescent proteins, enzymes, and enhancing agents (e.g., paramagnetic ions).

In certain embodiments, the B7-H3-targeting agent is conjugated to a contrast agent. Exemplary contrast agents include ultrasound contrast agents (e.g. SonoVue microbubbles comprising sulphur hexafluoride, Optison microbubbles comprising an albumin shell and octafluoropropane gas core, Levovist microbubbles comprising a lipid/galactose shell and an air core, Perflexane lipid microspheres comprising perfluorocarbon microbubbles, and Perflutren lipid microspheres comprising octafluoropropane encapsulated in an outer lipid shell), magnetic resonance imaging (MRI) contrast agents (e.g., gadodiamide, gadobenic acid, gadopentetic acid, gadoteridol, gadofosveset, gadoversetamide, gadoxetic acid), and radiocontrast agents, such as for computed tomography (CT), radiography, or fluoroscopy (e.g., diatrizoic acid, metrizoic acid, iodamide, iotalamic acid, ioxitalamic acid, ioglicic acid, acetrizoic acid, iocarmic acid, methiodal, diodone, metrizamide, iohexol, ioxaglic acid, iopamidol, iopromide, iotrolan, ioversol, iopentol, iodixanol, iomeprol, iobitridol, ioxilan, iodoxamic acid, iotroxic acid, ioglycamic acid, adipiodone, iobenzamic acid, iopanoic acid, iocetamic acid, sodium iopodate, tyropanoic acid, and calcium iopodate).

In one embodiment, the B7-H3-targeting agent is conjugated to a microbubble that can be used as a contrast agent for ultrasound imaging. Microbubbles are composed of a shell encapsulating a gas core. The shell may be formed from any suitable material, including but not limited to proteins (e.g., albumin), polysaccharides (e.g., galactose), lipids (such as phospholipids), polymers, surfactants, and combinations thereof. Any suitable gas core can be used in microbubbles, including, but not limited to, air, octafluoropropane, perfluorocarbon, sulfur hexafluoride, or nitrogen. The microbubbles oscillate and vibrate when a sonic energy field is applied and reflect ultrasound waves. The gas core determines the echogenicity (i.e., the ability of an object to reflect ultrasound waves) of the microbubble.

The average diameter of a microbubble is typically between about 1 μm and about 25 μm. In general, microbubbles have a diameter ranging between about 1 μm and about 10 μm on average, and more preferably between about 1 μm and 5 μm, 1 μm and 4 μm, 1 μm and 3 μm, 1 μm and about 2 μm, 2 μm and 5 μm, 2 μm and 4 μm, 2 μm and 3 μm, 3 μm and 5 μm, 3 μm and 4 μm, or about 1 μm, 2 μm, 2.5 μm, 3 μm, 3.5 μm, or 4 μm on average.

Various types of microbubbles are commercially available, including but not limited to OPTISON microbubbles (made by GE Healthcare), the first microbubble approved by the Food and Drug Administration (FDA), which have an albumin shell and an octafluoropropane (C₃F₈) gas core; LEVOVIST microbubbles (made by Schering AG), the second FDA-approved microbubble, which have a palmitic acid/galactose shell and an air core; ALBUNEX microbubbles (made by Molecular Biosystems), which have an albumin shell and an air core; SONOVUE microbubbles (made by Bracco Diagnostics, Inc.), which have a sulfur hexafluoride (SF₆) gas core that is stabilized in aqueous dispersion of a monolayer of phospholipids; SONOZOID microbubbles (made by Schering AG), which have a perfluorocarbon gas core and a lipid shell; SONOVIST microbubbles (made by Schering AG), which have a cyanoacrylate polymer shell and an air core; DEFINITY microbubbles (made by DuPont Pharmaceuticals), which have a lipid/surfactant shell and an octafluoropropane (C₃F) gas core; and CARDIOSPHERE microbubbles (made by POINT Biomedical Corporation), which have a polyactide polymer shell and a nitrogen gas core.

Microbubbles have a high degree of echogenicity. The echogenicity difference between the gas in the microbubbles and the soft tissue surroundings of the body is large. Thus, ultrasonic imaging using microbubble contrast agents enhances the ultrasound backscatter, or reflection of the ultrasound waves, to produce a unique sonogram with increased contrast due to the high echogenicity difference.

B7-H3-targeting agents can be attached to the shell surface of microbubbles. Following administration to a patient (such as by intravenous injection), microbubbles carrying the B7-H3-targeting agents (i.e., B7-H3-targeted microbubbles) accumulate at tissue sites that overexpress B7-H3 resulting in a local increase in the ultrasound imaging signal. Microbubbles stay predominantly within the vascular compartment after intravenous injection. Thus, microbubbles can be used, for example, to detect B7-H3 antigen on the surface of breast tumor neovasculature or epithelium or precancerous lesions that are present in early stage breast cancer.

The surface of a microbubble can be functionalized in any suitable manner for binding of a B7-H3-targeting agent. For example, the microbubble surface can be functionalized with maleimide to permit conjugation of a cysteine thiol of the antibody to the microbubble surface. Alternatively, the microbubble surface can be coated with streptavidin to allow binding of biotinylated B7-H3-targeting agents. Streptavidin-coated microbubbles are commercially available from, e.g., VisualSonics (Toronto, Canada) and Akadeum Life Sciences (Ann Arbor, Mich.). Any other suitable binding pair can be similarly used, as will be apparent to those of skill in the art.

In other embodiments, the diagnostic agent is a radioactive metal, paramagnetic ion, or other diagnostic cation. In this case, the B7-H3-targeting agent can be conjugated to a chelating group for binding cations. Exemplary chelating agents include ethylenediaminetetraacetic acid (EDTA), diethylenetriaminepentaacetic acid (DTPA), 1,4,7,10-tetraazacyclododecane-1,4,7,10-tetraacetic acid (DOTA), 1,4,7-triazacyclononane-N,N′,N″-triacetic acid (NOTA), NETA, p-bromoacetamido-benzyl-tetraethylaminetetraacetic acid (TETA), porphyrins, polyamines, crown ethers, bis-thiosemicarbazones, polyoximes, and like groups known to be useful for this purpose. Chelates are coupled to the B7-H3-targeting agents using standard chemistries, which then can be used to bind diagnostic isotopes such as ¹²⁵I, ¹³¹I, ¹²³I, ¹²⁴I, ⁶²Cu, ⁶⁴Cu, ¹⁸F, ¹¹¹In, ⁶⁷Ga, ⁶⁸Ga, ^{99m}Tc, ²²³Ra, ¹¹C, ¹³N, ¹⁵O, and ⁷⁶Br for radioimaging. The same chelates, when complexed with non-radioactive metals, such as manganese, iron and gadolinium are useful for MRI, when used along with the B7-H3-targeting agents.

Diagnostic agents comprising ¹⁸F or ¹¹C can be used in PET imaging. For example, a B7-H3-targeting agent can be isotopically labeled with ¹⁸F or ¹¹C or conjugated to ¹⁸F or ¹¹C-labeled compounds for use in PET imaging.

Administration of B7-H3-Targeted Imaging Agents

Preferably, a detectably effective amount of a B7-H3-targeted imaging agent (e.g., a B7-H3-targeting agent conjugated to a diagnostic agent) is administered to a subject; that is, an amount that is sufficient to yield an acceptable image using the imaging equipment that is available for clinical use. A detectably effective amount of the B7-H3-targeted imaging agent may be administered in more than one injection if needed. The detectably effective amount of the B7-H3-targeted imaging agent needed for an individual may vary according to factors such as the degree of binding of the imaging agent to breast tissue, the age, sex, and weight of the individual, and the particular medical imaging method used. Optimization of such factors is within the level of skill in the art.

Imaging with B7-H3-targeted imaging agents can be used in assessing efficacy of therapeutic drugs in treating breast cancer. For example, images can be acquired after treatment with an anti-cancer therapy to determine if the individual is responding to treatment. In a subject with breast cancer, imaging with a B7-H3-targeted imaging agent can be used to evaluate whether a tumor is shrinking or growing. Further, the extent of cancerous disease (stage of cancer progression) can be determined to aid in determining prognosis and evaluating optimal strategies for treatment (e.g., surgery, radiation, or chemotherapy).

Additionally, B7-H3-targeted imaging agents can be used in image-guided surgery. Breast cells or tissue of interest can be contacted with a B7-H3-targeted imaging agent, such that the B7-H3-targeted imaging agent binds to any B7-H3 antigen present on the surface of cells or tissue (e.g., B7-H3 overexpressed on breast tumor neovasculature, breast tumor epithelium, cancerous cells, or precancerous lesions). Imaging of tissues labeled with the B7-H3-targeted imaging agent in this way can be used, for example, for detection of pathology, tumor margin delineation, evaluation of the completeness of resection, and evaluation of the efficacy of treatment.

For example, B7-H3-targeted imaging agents may be used, pre-operatively for visualization of breast tumor neovasculature, breast tumor epithelium, cancerous cells, or precancerous lesions to assist surgical planning. Alternatively or additionally, B7-H3-targeted imaging agents may be used intra-operatively to provide image-guidance to increase the probability of success in achieving total resection of cancerous tissue and minimize damage to adjacent structures. Intraoperatively acquired images can be used, for example, to improve microsurgical treatment of tumors and other lesions. Real-time imaging can be used to allow continuous monitoring during surgery.

Imaging of breast tissue can be performed using any appropriate imaging technique, including, but not limited to, ultrasound imaging, positron emission tomography, single photon emission computed tomography, magnetic resonance imaging, computed tomography, optical imaging, photoacoustic imaging, fluoroscopy, and fluorescence imaging. The choice of imaging technique will depend on the selection of a particular B7-H3 imaging agent. As described above, B7-H3-imaging agents may comprise contrast agents (e.g., ultrasound, MRI, or radiocontrast agents), photoactive agents, radioisotopes, nonradioactive isotopes, dyes, fluorescent compounds or proteins, chemiluminescent compounds, bioluminescent proteins, enzymes, or enhancing agents suitable for various imaging methods.

Images of breast tissue can be acquired, for example, using an ultrasound scanner, a magnetic resonance imaging instrument (MRI scanner), an X-ray source with film or a detector (e.g., conventional or digital radiography system), an X-ray computed tomography (CT) or computed axial tomography (CAT) scanner, a gamma camera, or a positron emission tomography (PET) scanner. Various medical imaging systems have been developed for open surgery as well as for laparoscopic, thoracoscopic, and robot-assisted surgery and can be used in the practice of the invention. Conventional laparoscopes and endoscopes can be equipped with a photodetector (e.g., camera or CCD detector) to provide guidance during medical procedures. Fiber-optic imaging systems can also be used, which include portable handheld microscopes, flexible endoscopes, and microendoscopes. For example, an illumination source can be added to such devices to allow fluorescence imaging. A miniaturized ultrasound transducer can be added to the tip of a laparoscope or catheter for intravascular ultrasound (IVUS) imaging. Miniaturized imaging systems can be used that allow imaging inside small cavities and constricted spaces. In addition, miniaturized imaging devices (e.g., microendoscopes) may be implanted within a subject for long-term imaging studies. In addition, a camera may be used to take both photographic images of a subject and to detect signals from a B7-H3-targeted imaging agent, so that photographic images of the subject and images of the signals from the bound B7-H3-targeted imaging agent can be superimposed to allow regions containing the bound B7-H3-targeted imaging agent to be mapped to the subject's anatomy. For a review of medical imaging methods and their use in image-guided surgery, see, e.g., Tartar et al. Breast Cancer Imaging: A Multidisciplinary, Multimodality Approach (Mosby, 2008); Stavros et al. Breast Ultrasound (LWW, 2003); Kopans Breast Imaging (LWW, Third edition, 2006); Breast MRI: Diagnosis and Intervention (L. Liberman ed., Springer, 2005); Wolbarst et al. Medical Imaging: Essentials for Physicians (Wiley-Blackwell, 2013); Darby et al. Oxford Handbook of Medical Imaging (Oxford University Press, 2012); P. Suetens Fundamentals of Medical Imaging (Cambridge University Press, 2^{nd} edition, 2009); J. L. Prince et al. Medical Imaging Signals and Systems (Pearson; 2^{nd} edition, 2014); Tang et al. (2015) IEEE Trans Biomed Circuits Syst. 9(6):767-776; Khuri-Yakub et al. (2010) Conf. Proc. IEEE Eng. Med. Biol. Soc. 2010:5987-90; Gray et al. (2012) Biomed. Opt. Express. 3(8): 1880-1890; Flusberg et al. (2005) Nat. Methods 2(12):941-950; Choyke et al. (2012) IEEE J. Sel. Top. Quantum. Electron. 18(3):1140-1146; Gray et al. (2012) Proc. SPIE February 3: 8207; Vahrmeijer et al. (2013) Nat. Rev. Clin. Oncol. 10:507-518; Braks et al. (2013) Methods Mol. Biol. 923:353-368; Yong et al. (2011) Diabetes 60(5): 1383-1392; Wilson et al. (2008) J. Vis. Exp. May 2(14) pii: 740; Engelsman et al. (2009) J. Biomed. Mater. Res B Appl Biomater. 88(1): 123-129; Franke-Fayard et al. (2006) Nat. Protoc. 1(1):476-485; Rehemtulla et al. (2000) Neoplasia. 2(6):491-495; and Close et al. (2011) Sensors 11:180-206; herein incorporated by reference in their entireties.

Additionally, images may be recorded by any suitable method. For example, a CCD image sensor, CMOS image sensor, or digital camera may be used to capture images. The image may be a still photo or a video in any format (e.g., bitmap, Graphics Interchange Format, JPEG file interchange format, TIFF, or mpeg). Alternatively, images may be captured by an analog camera and converted into an electronic form.

B7-H3-Targeted Therapeutic Agents

B7-H3-targeting agents can also be used to target therapeutic agents to the location of B7-H3-expressing breast tumors, cancerous cells, or precancerous lesions to directly treat breast cancer in a subject. B7-H3-targeting agents can be conjugated to one or more therapeutic agents, such as, but not limited to, drugs, toxins, radioisotopes, immunomodulators, angiogenesis inhibitors, therapeutic enzymes, and cytotoxic or pro-apoptotic agents for treatment of breast cancer.

For example, a B7-H3-targeting agent can be conjugated to one or more chemotherapeutic agents such as, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

Alternatively or additionally, a B7-H3-targeting agent can be conjugated to, one or more tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; and/or small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar).

In another example, the B7-H3-targeting agent can be conjugated to a hormonal blocking therapeutic agent for treatment of a breast cancer depending on estrogen for growth (e.g., cancer expressing estrogen receptors (ER+ cancer)). For example, the anti-B7-H3 antibody can be conjugated to a drug that blocks ER receptors (e.g. tamoxifen) or a drug that blocks the production of estrogen, such as an aromatase inhibitor (e.g. anastrozole, or letrozole).

In another example, the B7-H3-targeting agent can be conjugated to a toxin. The toxin can be of animal, plant or microbial origin. Exemplary toxins include Pseudomonas exotoxin, ricin, abrin, Staphylococcal enterotoxin-A, pokeweed antiviral protein, gelonin, diphtheria toxin, and Pseudomonas endotoxin.

In a further example, the B7-H3-targeting agent can be conjugated to an immunomodulator, such as a cytokine, a lymphokine, a monokine, a stem cell growth factor, a lymphotoxin (LT), a hematopoietic factor, a colony stimulating factor (CSF), an interferon (IFN), parathyroid hormone, thyroxine, insulin, proinsulin, relaxin, prorelaxin, follicle stimulating hormone (FSH), thyroid stimulating hormone (TSH), luteinizing hormone (LH), hepatic growth factor, prostaglandin, fibroblast growth factor, prolactin, placental lactogen, OB protein, a transforming growth factor (TGF), such as TGF-α or TGF-β, insulin-like growth factor (IGF), erythropoietin, thrombopoietin, a tumor necrosis factor (TNF) such as TNF-α or TNF-β, a mullerian-inhibiting substance, mouse gonadotropin-associated peptide, inhibin, activin, vascular endothelial growth factor, integrin, granulocyte-colony stimulating factor (G-CSF), granulocyte macrophage-colony stimulating factor (GM-CSF), an interferon such as interferon-α, interferon-β, or interferon-γ, S1 factor, an interleukin (IL) such as IL-1, IL-1cc, IL-2, IL-3, IL-4, IL-5, IL-6, IL-7, IL-8, IL-9, IL-10, IL-11, IL-12, IL-13, IL-14, IL-15, IL-16, IL-17, IL-18 IL-21 or IL-25, LIF, kit-ligand, FLT-3, angiostatin, thrombospondin, endostatin, and LT.

In another embodiment, the B7-H3-targeting agent is conjugated to a radioactive isotope. Particularly useful therapeutic radionuclides include, but are not limited to ¹¹In, ¹⁷⁷Lu, ²¹²Bi, ²¹³Bi, ²¹¹At, ⁶²Cu, ⁶⁴Cu, ⁶⁷Cu, ⁹⁰Y, ¹²³I, ¹²⁵I, ¹³¹I, ³²P, ³³P, ⁷⁷Br, ⁴⁷Sc, ¹¹¹Ag, ⁶⁷Ga, ¹⁴²Pr, ¹⁵³Sm, ¹⁶¹Tb, ¹⁶⁶Dy, ¹⁶⁶Ho, ¹⁸⁶Re, ¹⁸⁸Re, ¹⁸⁹Re, ²¹²Pb, ²²³Ra, ²²⁵Ac, ⁵⁹Fe, ⁷⁵Se, ⁷⁷As, ⁸⁹Sr, ⁹⁹Mo, ¹⁰⁵Rh, ¹⁰⁹Pd, ¹⁴³Pr, ¹⁴⁹Pm, ¹⁶⁹Er, ¹⁹⁴Ir, ¹⁹⁸Au, and ¹⁹⁹Au.

In certain embodiments, the therapeutic radionuclide has a decay energy in the range of 20 to 6,000 keV (e.g, 60 to 200 keV for an Auger emitter, 100-2,500 keV for a beta emitter, and 4,000-6,000 keV for an alpha emitter). In one embodiment, the radionuclide is an Auger-emitter (e.g., Co-58, Ga-67, Br-80m, Tc-99m, Rh-103m, Pt-109, In-111, Sb-119, 1-125, Ho-161, Os-189m and Ir-192). In another embodiment, the radionuclide is an alpha-emitter (e.g., Dy-152, At-211, Bi-212, Ra-223, Rn-219, Po-215, Bi-211, Ac-225, Fr-221, At-217, Bi-213 and Fm-255).

Additional therapeutic radioisotopes include ¹¹C, ¹³N, ¹⁵O, ⁷⁵Br, ¹⁹⁸Au, ²²⁴Ac, ¹²⁶I, ¹³³I, ⁷⁷Br, ^{113m}In, ⁹⁵Ru, ⁹⁷Ru, ¹⁰³Ru, ¹⁰⁵Ru, ¹⁰⁷Hg, ²⁰³Hg, ^{121m}Te, ^{122m}Te, ¹⁶⁵Tm, ¹⁶⁷Tm, ¹⁶⁸Tm, ¹⁹⁷Pt, ¹⁰⁹Pd, ¹⁰⁵Rh, ¹⁴²Pr, ¹⁴³Pr, ¹⁶¹Tb, ¹⁶⁶Ho, ¹⁹⁹Au, ⁵⁷Co, ⁵¹Cr, ⁵⁹Fe, ⁷⁵Se, ²⁰¹Tl, ²²⁵Ac, ⁷⁶Br, ¹⁶⁹Yb, and the like.

B7-H3-targeting agents may also be conjugated to a boron addend-loaded carrier for thermal neutron activation therapy. For example, boron addends such as carboranes, can be attached to B7-H3-targeting agents. Carboranes can be prepared with carboxyl functions on pendant side chains, as is well-known in the art. Attachment of carboranes to a carrier, such as aminodextran, can be achieved by activation of the carboxyl groups of the carboranes and condensation with amines on the carrier. The intermediate conjugate is then conjugated to the B7-H3-targeting agent. After administration of the B7-H3-targeting agent conjugate, a boron addend is activated by thermal neutron irradiation and converted to radioactive atoms which decay by alpha-emission to produce highly toxic, short-range effects.

C. Pharmaceutical Compositions

Bioconjugates of a B7-H3-targeting agent (e.g., conjugated to one or more diagnostic agents or therapeutic agents, or a combination thereof) can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerine, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition of the invention can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the B7-H3-targeting agent bioconjugate (e.g., conjugated to one or more diagnostic agents or therapeutic agents, or a combination thereof), or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, N.J.); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of the B7-H3-targeting agent bioconjugate (e.g., when contained in a drug delivery system) in the composition will vary depending on a number of factors, but will optimally be a therapeutically effective dose when the composition is in a unit dosage form or container (e.g., a vial). A therapeutically effective dose can be determined experimentally by repeated administration of increasing amounts of the composition in order to determine which amount produces a clinically desired endpoint.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, N.J. (1998), and Kibbe, A. H., Handbook of Pharmaceutical Excipients, 3rd Edition, American Pharmaceutical Association, Washington, D.C. , 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for oral, ocular, or localized delivery.

The pharmaceutical preparations herein can also be housed in a syringe, an implantation device, or the like, depending upon the intended mode of delivery and use. Preferably, the compositions comprising a B7-H3-targeting agent bioconjugate are in unit dosage form, meaning an amount of a conjugate or composition of the invention appropriate for a single dose, in a premeasured or pre-packaged form.

The compositions herein may optionally include one or more additional agents, such as drugs for treating cancer or other medications used to treat a subject for a condition or disease. Compounded preparations may include a B7-H3-targeting agent bioconjugate (e.g, conjugated to one or more diagnostic agents or therapeutic agents, or a combination thereof) and one or more drugs for treating breast cancer, such as, but not limited to, chemotherapy, immunotherapy, biologic or targeted therapy agents. Alternatively, such agents can be contained in a separate composition from the composition comprising a B7-H3-targeting agent bioconjugate and co-administered concurrently, before, or after the composition comprising the B7-H3-targeting agent bioconjugate of the invention.

D. Administration of B7-H3-Targeted Therapeutic Agents

The methods of the invention can be used for treating a subject for breast cancer. Thus, B7-H3-targeted therapeutic agents comprising a B7-H3-targeting agent conjugated to an anti-cancer therapeutic agent (or B7-H3-targeted theranostic agent also conjugated to a diagnostic agent) can be used to treat, for example, breast cancer expressing the B7-H3 antigen, including a tumor, cancer, or metastasis that is progressing, worsening, stabilized or in remission as well as precancerous lesions.

At least one therapeutically effective cycle of treatment with a B7-H3-targeted therapeutic agent (e.g., a B7-H3-targeting agent conjugated to an anti-cancer therapeutic agent) will be administered to a subject for treatment of breast cancer. By “therapeutically effective dose or amount” of a B7-H3-targeted therapeutic agent is intended an amount that when administered brings about a positive therapeutic response with respect to treatment of an individual for cancer. Of particular interest is an amount of a B7-H3-targeted therapeutic agent that provides an anti-tumor effect, as defined herein. By “positive therapeutic response” is intended the individual undergoing the treatment according to the invention exhibits an improvement in one or more symptoms of the cancer for which the individual is undergoing therapy.

Thus, for example, a “positive therapeutic response” would be an improvement in the disease in association with the therapy, and/or an improvement in one or more symptoms of the disease in association with the therapy. Therefore, for example, a positive therapeutic response would refer to one or more of the following improvements in the disease: (1) reduction in tumor size; (2) reduction in the number of cancer cells; (3) inhibition (i.e., slowing to some extent, preferably halting) of tumor growth; (4) inhibition (i.e., slowing to some extent, preferably halting) of cancer cell infiltration into peripheral organs; (5) inhibition (i.e., slowing to some extent, preferably halting) of tumor metastasis; and (6) some extent of relief from one or more symptoms associated with the cancer. Such therapeutic responses may be further characterized as to degree of improvement. Thus, for example, an improvement may be characterized as a complete response. By “complete response” is documentation of the disappearance of all symptoms and signs of all measurable or evaluable disease confirmed by physical examination, laboratory, ultrasound, nuclear, radiographic studies (i.e., CT (computer tomography), and/or MRI (magnetic resonance imaging)), and other non-invasive procedures repeated for all initial abnormalities or sites positive at the time of entry into the study. Alternatively, an improvement in the disease may be categorized as being a partial response. By “partial response” is intended a reduction of greater than 50% in the sum of the products of the perpendicular diameters of all measurable lesions when compared with pretreatment measurements.

In certain embodiments, multiple therapeutically effective doses of compositions comprising a B7-H3-targeted therapeutic agent (e.g., a B7-H3-targeting agent conjugated to an anti-cancer therapeutic agent), and/or one or more other therapeutic agents, such as other drugs for treating cancer, or other medications will be administered. The compositions of the present invention are typically, although not necessarily, administered orally, via injection (subcutaneously, intravenously, or intramuscularly), by infusion, or locally. Additional modes of administration are also contemplated, such as intra-arterial, intraperitoneal, pulmonary, nasal, topical, transdermal, intralesion, intrapleural, intraparenchymatous, rectal, transdermal, transmucosal, intrathecal, pericardial, intra-arterial, intraocular, and so forth. When administering the B7-H3-targeted therapeutic agent by injection, the administration may be by continuous infusion or by single or multiple boluses.

The preparations according to the invention are also suitable for local treatment. In a particular embodiment, a composition of the invention is used for localized delivery of a B7-H3-targeted therapeutic agent for the treatment of breast cancer. For example, compositions may be administered directly into a breast tumor or cancerous cells. Administration may be by perfusion through a regional catheter or direct intralesional injection.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising a B7-H3-targeted therapeutic agent and other agents may be administered using the same or different routes of administration in accordance with any medically acceptable method known in the art.

In another embodiment, the pharmaceutical compositions comprising a B7-H3-targeted therapeutic agent and/or other agents are administered prophylactically, e.g., to prevent cancer progression in breast tissue. Such prophylactic uses will be of particular value for subjects with a potentially precancerous or premalignant condition (e.g., precancerous lesions, dysplasia or benign neoplasia), or who have a genetic predisposition to developing breast cancer.

In another embodiment of the invention, the pharmaceutical compositions comprising a B7-H3-targeted therapeutic agent and/or other agents are in a sustained-release formulation, or a formulation that is administered using a sustained-release device. Such devices are well known in the art, and include, for example, transdermal patches, and miniature implantable pumps that can provide for drug delivery over time in a continuous, steady-state fashion at a variety of doses to achieve a sustained-release effect with a non-sustained-release pharmaceutical composition.

The invention also provides a method for administering a conjugate comprising a B7-H3-targeted therapeutic agent as provided herein to a patient suffering from breast cancer that is responsive to treatment with a B7-H3-targeted therapeutic agent contained in the conjugate or composition. The method comprises administering, via any of the herein described modes, a therapeutically effective amount of the conjugate or drug delivery system, preferably provided as part of a pharmaceutical composition. The method of administering may be used to treat any cancer that is responsive to treatment with a B7-H3-targeted therapeutic agent. More specifically, the compositions herein are effective in treating breast cancer.

Those of ordinary skill in the art will appreciate which conditions a B7-H3-targeted therapeutic agent can effectively treat. The actual dose to be administered will vary depending upon the age, weight, and general condition of the subject as well as the severity of the condition being treated, the judgment of the health care professional, and conjugate being administered. Therapeutically effective amounts can be determined by those skilled in the art, and will be adjusted to the particular requirements of each particular case.

Generally, a therapeutically effective amount will range from about 0.50 mg to 5 grams of a B7-H3-targeted therapeutic agent inhibitor daily, more preferably from about 5 mg to 2 grams daily, even more preferably from about 7 mg to 1.5 grams daily. Preferably, such doses are in the range of 10-600 mg four times a day (QID), 200-500 mg QID, 25-600 mg three times a day (TID), 25-50 mg TID, 50-100 mg TID, 50-200 mg TID, 300-600 mg TID, 200-400 mg TID, 200-600 mg TID, 100 to 700 mg twice daily (BID), 100-600 mg BID, 200-500 mg BID, or 200-300 mg BID. The amount of compound administered will depend on the potency of the specific B7-H3-targeted therapeutic agent and the magnitude or effect desired and the route of administration.

A purified B7-H3-targeted therapeutic agent (again, preferably provided as part of a pharmaceutical preparation) can be administered alone or in combination with one or more other therapeutic agents, such as chemotherapy, immunotherapy, biologic or targeted therapy agents, or other medications used to treat a particular condition or disease according to a variety of dosing schedules depending on the judgment of the clinician, needs of the patient, and so forth. The specific dosing schedule will be known by those of ordinary skill in the art or can be determined experimentally using routine methods. Exemplary dosing schedules include, without limitation, administration five times a day, four times a day, three times a day, twice daily, once daily, three times weekly, twice weekly, once weekly, twice monthly, once monthly, and any combination thereof. Preferred compositions are those requiring dosing no more than once a day.

A B7-H3-targeted therapeutic agent can be administered prior to, concurrent with, or subsequent to other agents. If provided at the same time as other agents, the B7-H3-targeted therapeutic agent can be provided in the same or in a different composition. Thus, the B7-H3-targeted therapeutic agent and other agents can be presented to the individual by way of concurrent therapy. By “concurrent therapy” is intended administration to a subject such that the therapeutic effect of the combination of the substances is caused in the subject undergoing therapy. For example, concurrent therapy may be achieved by administering a dose of a pharmaceutical composition comprising a B7-H3-targeted therapeutic agent and a dose of a pharmaceutical composition comprising at least one other agent, such as another drug for treating cancer, which in combination comprise a therapeutically effective dose, according to a particular dosing regimen. Similarly, the B7-H3-targeted therapeutic agent and one or more other therapeutic agents can be administered in at least one therapeutic dose. Administration of the separate pharmaceutical compositions can be performed simultaneously or at different times (i.e., sequentially, in either order, on the same day, or on different days), as long as the therapeutic effect of the combination of these substances is caused in the subject undergoing therapy.

Where a subject undergoing therapy in accordance with the previously mentioned dosing regimens exhibits a partial response or a relapse following a prolonged period of remission, subsequent courses of concurrent therapy may be needed to achieve complete remission of the disease. Thus, subsequent to a period of time off from a first treatment period, a subject may receive one or more additional treatment periods with the B7-H3-targeted therapeutic agent. Such a period of time off between treatment periods is referred to herein as a time period of discontinuance. It is recognized that the length of the time period of discontinuance is dependent upon the degree of tumor response (i.e., complete versus partial) achieved with any prior treatment periods of concurrent therapy with these therapeutic agents.

Additionally, treatment with a B7-H3-targeted therapeutic agent may be combined with any other medical treatment for cancer, such as, but not limited to, surgery, radiation therapy, chemotherapy, hormonal therapy, immunotherapy, or molecularly targeted or biologic therapy. Any combination of these other medical treatment methods with a B7-H3-targeted therapeutic agent may be used to effectively treat cancer in a subject.

For example, treatment with a B7-H3-targeted therapeutic agent may be combined with chemotherapy with one or more chemotherapeutic agents such as, but not limited to, abitrexate, adriamycin, adrucil, amsacrine, asparaginase, anthracyclines, azacitidine, azathioprine, bicnu, blenoxane, busulfan, bleomycin, camptosar, camptothecins, carboplatin, carmustine, cerubidine, chlorambucil, cisplatin, cladribine, cosmegen, cytarabine, cytosar, cyclophosphamide, cytoxan, dactinomycin, docetaxel, doxorubicin, daunorubicin, ellence, elspar, epirubicin, etoposide, fludarabine, fluorouracil, fludara, gemcitabine, gemzar, hycamtin, hydroxyurea, hydrea, idamycin, idarubicin, ifosfamide, ifex, irinotecan, lanvis, leukeran, leustatin, matulane, mechlorethamine, mercaptopurine, methotrexate, mitomycin, mitoxantrone, mithramycin, mutamycin, myleran, mylosar, navelbine, nipent, novantrone, oncovin, oxaliplatin, paclitaxel, paraplatin, pentostatin, platinol, plicamycin, procarbazine, purinethol, ralitrexed, taxotere, taxol, teniposide, thioguanine, tomudex, topotecan, valrubicin, velban, vepesid, vinblastine, vindesine, vincristine, vinorelbine, VP-16, and vumon.

In another example, treatment with a B7-H3-targeted therapeutic agent may be combined with targeted therapy with one or more small molecule inhibitors or monoclonal antibodies such as, but not limited to, tyrosine-kinase inhibitors, such as Imatinib mesylate (Gleevec, also known as STI-571), Gefitinib (Iressa, also known as ZD1839), Erlotinib (marketed as Tarceva), Sorafenib (Nexavar), Sunitinib (Sutent), Dasatinib (Sprycel), Lapatinib (Tykerb), Nilotinib (Tasigna), and Bortezomib (Velcade); Janus kinase inhibitors, such as tofacitinib; ALK inhibitors, such as crizotinib; Bcl-2 inhibitors, such as obatoclax and gossypol; PARP inhibitors, such as Iniparib and Olaparib; PI3K inhibitors, such as perifosine; VEGF Receptor 2 inhibitors, such as Apatinib; AN-152 (AEZS-108) doxorubicin linked to [D-Lys(6)]-LHRH; Braf inhibitors, such as vemurafenib, dabrafenib, and LGX818; MEK inhibitors, such as trametinib; CDK inhibitors, such as PD-0332991 and LEE011; Hsp90 inhibitors, such as salinomycin; small molecule drug conjugates, such as Vintafolide; serine/threonine kinase inhibitors, such as Temsirolimus (Torisel), Everolimus (Afinitor), Vemurafenib (Zelboraf), Trametinib (Mekinist), and Dabrafenib (Tafinlar); and monoclonal antibodies, such as Rituximab (marketed as MabThera or Rituxan), Trastuzumab (Herceptin), Alemtuzumab, Cetuximab (marketed as Erbitux), Panitumumab, Bevacizumab (marketed as Avastin), and Ipilimumab (Yervoy).

In a further example, treatment with a B7-H3-targeted therapeutic agent may be combined with immunotherapy, including, but not limited to, using any of the following: a cancer vaccine (e.g., E75 HER2-derived peptide vaccine, nelipepimut-S (NeuVax)), antibody therapy (e.g., Trastuzumab, Ado-trastuzumab emtansine), cytokine therapy (e.g., interferons, including type I (IFNα and IFNβ), type II (IFNγ) and type III (IFNλ) and interleukins, including interleukin-2 (IL-2)), adjuvant immunochemotherapy (e.g., polysaccharide-K), adoptive T-cell therapy, and immune checkpoint blockade therapy.

E. Immunoassays Using Anti-B7-H3 Antibodies

Anti-B7-H3 antibodies can also be used for detection of breast cancer in vitro. For example, anti-B7-H3 antibodies can be used to detect the presence of the B7-H3 antigen in breast tissue from biopsy samples. Anti-B7-H3 antibodies can also be used to detect and measure the amount of B7-H3 antigen in breast tissue samples using immunoassay techniques such as immunohistochemistry (IHC), enzyme-linked immunosorbent assay (ELISA), radioimmunoassays (RIA), “sandwich” immunoassays, fluorescent immunoassays, enzyme multiplied immunoassay technique (EMIT), capillary electrophoresis immunoassays (CEIA) immunoprecipitation assays, and western blotting, the procedures of which are well known in the art (see, e.g., Ausubel et al, eds, 1994, Current Protocols in Molecular Biology, Vol. 1, John Wiley & Sons, Inc., New York, which is incorporated by reference herein in its entirety).

Anti-B7-H3 antibodies may be used in diagnostic assays to detect the presence or for quantification of the B7-H3 antigen in a breast tissue sample. Such a diagnostic assay may comprise at least two steps: (i) contacting the B7-H3 antigen from a breast tissue sample with an anti-B7-H3 antibody, and (ii) quantifying the antibody bound to the B7-H3 antigen. The method may additionally involve a preliminary step of attaching the antibody, either covalently, electrostatically, or reversibly, to a solid support, before subjecting the bound antibody to the sample, as defined above and elsewhere herein.

Various diagnostic assay techniques are known in the art, such as competitive binding assays, direct or indirect sandwich assays and immunoprecipitation assays conducted in either heterogeneous or homogenous phases (Zola, Monoclonal Antibodies: A Manual of Techniques, CRC Press, Inc., (1987), pp 147-158). The anti-B7-H3 antibodies used in the diagnostic assays can be labeled with a detectable moiety. The detectable moiety should be capable of producing, either directly or indirectly, a detectable signal. For example, the detectable moiety may be a radioisotope, such as ³H, ¹⁴C, ³²P, or ¹²⁵I, a fluorescent or chemiluminescent compound, such as fluorescein isothiocyanate, rhodamine, or luciferin, or an enzyme, such as alkaline phosphatase, beta-galactosidase, green fluorescent protein, or horseradish peroxidase. Any method known in the art for conjugating antibodies to the detectable moiety may be employed, including those methods described by Hunter et al., Nature, 144:945 (1962); David et al., Biochem., 13:1014 (1974); Pain et al., J. Immunol. Methods, 40:219 (1981); and Nygren, J. Histochem. and Cytochem., 30:407 (1982).

Immunoassays can be used to determine the presence or absence of B7-H3 antigen in a breast tissue sample as well as the quantity of the B7-H3 antigen in the sample. If the B7-H3 antigen is present in the sample, it will form an antibody-antigen complex with the anti-B7-H3 antibody, which specifically binds to the B7-H3 antigen under suitable incubation conditions. The amount of the antibody-antigen complex can be determined by comparing to a standard. A standard can be, e.g., a known compound or another protein known to be present in a sample.

Immunohistochemistry can be used to detect B7-H3 antigen on cancerous cells of a breast tissue section. For example, immunohistochemical staining with labeled anti-B7-H3 antibodies can be used to detect cancerous cells or precancerous lesions. Antibodies conjugated to enzymes, which catalyze color-producing reactions with chromogenic, fluorogenic, or chemiluminescent substrates (e.g., alkaline phosphatase or peroxidase), are commonly used. Alternatively, immunohistochemical staining can be performed with antibodies conjugated to fluorophores (e.g., fluorescein or rhodamine) to visualize biomarkers. See, e.g., Dabbs Diagnostic Immunohistochemistry: Theranostic and Genomic Applications, Saunders, 3^{rd} edition, 2010; Chu Modern Immunohistochemistry (Cambridge Illustrated Surgical Pathology) Cambridge University Press, 2009; Buchwalow et al. Immunohistochemistry: Basics and Methods, Springer, 1st Edition, 2010; and Ramos-Vara (2011) Methods Mol. Biol. 691:83-96; herein incorporated by reference in their entireties.

Flow cytometry can be used to detect multiple surface and intracellular markers simultaneously in whole cells and to distinguish populations of cells expressing different cellular markers. Typically, whole cells are incubated with antibodies that specifically bind to the markers. The antibodies can be labeled, for example, with a fluorophore, isotope, or quantum dot to facilitate detection of the markers. The cells are then suspended in a stream of fluid and passed through an electronic detection apparatus. (See, e.g., Shapiro Practical Flow Cytometry, Wiley-Liss, 4^{th} edition, 2003; Loken Immunofluorescence Techniques in Flow Cytometry and Sorting, Wiley, 2^{nd} edition, 1990; Flow Cytometry: Principles and Applications, (ed. Macey), Humana Press 1^{st} edition, 2007; herein incorporated by reference in their entireties.)

F. Production of B7-H3-Targeted Imaging and Therapeutic Agents

B7-H3-targeted imaging agents and therapeutic agents can be produced in any number of ways, all of which are well known in the art. In one embodiment, the B7-H3-targeted imaging agents and therapeutic agents are generated using recombinant techniques. One of skill in the art can readily determine nucleotide sequences that encode a B7-H3-targeted imaging agents and therapeutic agents using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding B7-H3-targeted imaging agents and therapeutic agents can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding anti-B7-H3 antibodies that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad Sci USA (1982) 79:6409.

Once coding sequences have been isolated and/or synthesized, they can be cloned into any suitable vector or replicon for expression. (See, also, Examples). As will be apparent from the teachings herein, a wide variety of vectors encoding modified polypeptides can be generated by creating expression constructs which operably link, in various combinations, polynucleotides encoding polypeptides having deletions or mutations therein.

Numerous cloning vectors are known to those of skill in the art, and the selection of an appropriate cloning vector is a matter of choice. Proteins have been successfully produced in variety of hosts, including plants, yeast, and bacteria (see, e.g., Wang et al. (2010) Protein Expr Purif 72:26-31; Brar et al. (2012) Mol Plant Microbe Interact 25:817-824; Huang et al. (2006) Appl Environ Microbiol 72:7748-7759; herein incorporated by reference).

Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), pU61 (Streptomyces), pUC6 (Streptomyces), YIp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555 (1987). Materials and methods for baculovirus/insect cell expression systems are commercially available in kit form from, inter alia, Invitrogen, San Diego Calif. (“MaxBac” kit).

Plant expression systems can also be used to produce the B7-H3-targeted imaging agents and therapeutic agents described herein. Generally, such systems use virus-based vectors to transfect plant cells with heterologous genes. For a description of such systems see, e.g., Porta et al., Mol. Biotech. (1996) 5:209-221; and Hackland et al., Arch. Virol. (1994) 139:1-22.

Viral systems, such as a vaccinia based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides and heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honey bee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the B7-H3-targeted imaging agents and therapeutic agents of the present invention are produced by growing host cells transformed by an expression vector described above under conditions whereby the protein of interest is expressed. The selection of the appropriate growth conditions is within the skill of the art.

In one embodiment, the transformed cells secrete the polypeptide product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted polypeptide product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant polypeptides substantially intact. Intracellular proteins can also be obtained by removing components from the cell wall or membrane, e.g., by the use of detergents or organic solvents, such that leakage of the polypeptides occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the polypeptide is expressed, culture conditions and any pre-treatment used.

Following disruption of the cells, cellular debris is removed, generally by centrifugation, and the intracellularly produced polypeptides are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining intracellular polypeptides involves affinity purification, such as by immunoaffinity chromatography using antibodies (e.g., previously generated antibodies), or by affinity chromatography using B7-H3 as a ligand. The choice of a suitable affinity resin is within the skill in the art. After affinity purification, the polypeptides can be further purified using conventional techniques well known in the art, such as by any of the techniques described above.

B7-H3-targeted imaging agents and therapeutic agents can be conveniently synthesized chemically, for example by any of several techniques that are known to those skilled in the peptide art. In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final polypeptide. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, Ill. 1984) and G. Barany and R. B. Merrifield, The Peptides: Analysis. Synthesis. Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis. Synthesis. Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, o-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

Polypeptide analogs can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

G. Kits

In yet another aspect, the invention provides kits comprising a B7-H3-targeted imaging agent that can be used to detect B7-H3 antigen on the surface of breast tumor neovasculature, breast tumor epithelium, cancerous cells, or precancerous lesions. Such kits can be used for detection, diagnosis, medical imaging, and/or treatment of breast cancer.

Compositions can be in liquid form or can be lyophilized. Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle).

The kit can further comprise a second container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery devices. The delivery device may be pre-filled with the compositions.

The kit can also comprise a package insert containing written instructions for methods of using the compositions comprising B7-H3-targeted imaging agents to detect and/or image cancerous cells or tumors in breast tissue expressing B7-H3, and methods of diagnosing and monitoring disease progression and therapeutic efficacy. The package insert can be an unapproved draft package insert or can be a package insert approved by the Food and Drug Administration (FDA) or other regulatory body.

In one embodiment, the kit comprises a B7-H3-targeted imaging agent comprising an anti-B7-H3 antibody conjugated to a microbubble and instructions for using the kit for imaging breast cancer using ultrasound.

### II. Experimental

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

Breast Cancer Detection by B7-H3-Targeted Ultrasound Molecular Imaging

Introduction:

Molecularly targeted contrast-enhanced ultrasound imaging is an emerging imaging strategy with large potential for improving diagnostic accuracy of conventional ultrasound imaging in earlier cancer detection (Kiessling et al. (2012) J. Nucl. Med. 53:345-348; Foygel et al. (2013) Gastroenterology 145:885-894; Bachawal et al. Cancer Res. 2013; 73:1689-1698). Ultrasound contrast agents are gas-filled echogenic microbubbles that can be further modified by adding binding ligands to the microbubble shell, which makes them firmly attach to molecular markers (Wen et al. (2014) J Nanosci. Nanotechnol. 14:190-209; Kiessling et al. (2014) Adv. Drug Deliv. Rev. 72:15-27). Because microbubbles are several micrometers in size, they remain exclusively within the vascular compartment (Kiessling et al., supra). This property of a purely intravascular contrast agent makes them particularly well suited for visualizing molecular markers expressed on the tumor neovasculature in various cancers, including breast cancer (Bachawal et al., supra; Bzyl et al. (2011) Eur. Radiol. 21:1988-1995). To achieve both high sensitivity and specificity in detecting breast cancer with ultrasound, it is of paramount importance to identify molecular markers as potential molecular imaging targets that are differentially expressed on the neovasculature of cancer compared with normal tissue, benign, and precursor breast lesions. Extensive research is under way aimed at identifying such cancer-specific vascular markers using various discovery techniques for both imaging and therapeutic purposes (Roesli et al. (2010) J. Proteomics 73:2219-2229).

Using a serial analysis of gene expression technique on isolated vascular endothelial cells, the transmembrane protein B7-H3, also known as CD276, was discovered as a novel tumor neovasculature-associated marker differentially expressed in murine and human colon, breast, and lung cancer xenografts grown in mice (Seaman et al. (2007) Cancer Cell 11:539-554). Recently, the B7-H3 protein was shown to be expressed in human breast cancer tissues (Turtoi et al. (2011) J. Proteome Res. 10:3160-3182). However, it is not known whether B7-H3 is differentially expressed on the neovasculature of breast cancer compared with benign, or precursor breast pathologies and normal breast tissue, which would make B7-H3 an attractive novel molecular imaging target for breast cancer detection using ultrasound.

The purpose of our study was twofold: First, to evaluate B7-H3 expression on the tumor neovasculature of breast cancer versus normal tissue, benign, and precursor breast lesions in a large-scale human IHC analysis study and, second, to assess feasibility of ultrasound molecular imaging using new B7-H3-targeted contrast microbubbles for breast cancer detection in a genetically engineered mouse model.

Materials and Methods

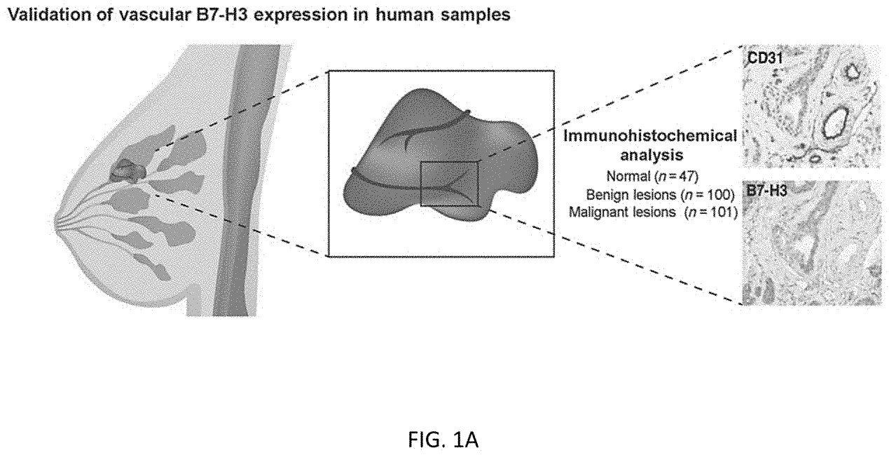

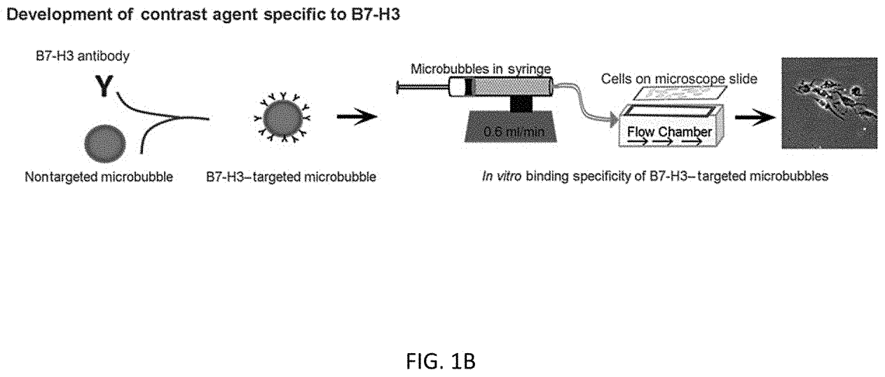

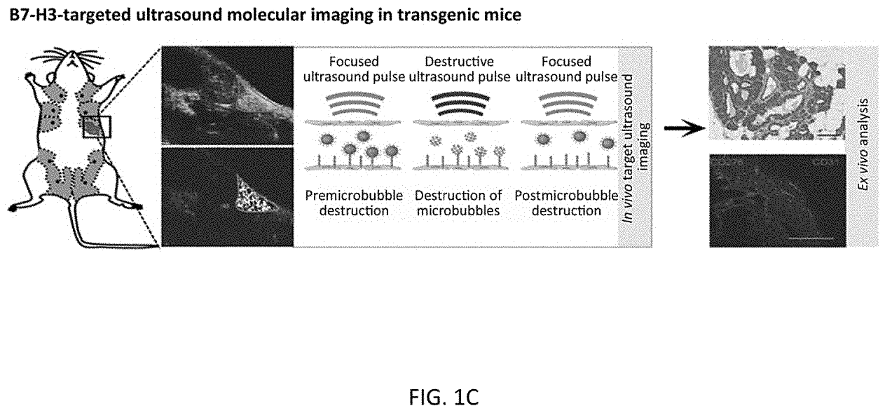

FIG. 1 summarizes the overall study design.

Collection of Human Breast Tissues

Human breast tissue samples were obtained retrospectively and were selected under an HIPAA compliant, Institutional Review Board-approved protocol to represent a range of normal tissue, benign and precursor lesions, and cancer tissues. A total of 248 samples were obtained, including 101 breast cancer samples, 100 benign or precursor pathologies, and 47 normal breast tissues (Table 1). Two hundred and nine samples were processed into a breast tissue microarray (TMA) using standardized protocols (Chandler et al. (2011) Methods Mol. Biol. 675:363-373). In brief, TMA cases were constructed from patient resection (surgical) tissues after characterization by a dedicated breast pathologist. Lesional areas were circled and 0.6 mm blocks were cored out from formalin-fixed paraffin-embedded tissue blocks by using a Beecher Tissue Microarrayer, and then slotted in a regular grid pattern into a blank recipient paraffin wax block. Thirty-nine whole-tissue samples of breast cancer were obtained from diagnostic large core needle biopsies. In these 39 whole-tissue cancer samples, benign tissues adjacent to breast cancer were used as intra-individual benign control tissues.

IHC Staining and Analysis of B7-H3 Expression in Human Breast Tissue Samples

IHC was performed on standard serial 5 mm sections of paraffin-embedded breast tissues using the Leica Bond Max automated platform (Leica Microsytems Inc.). This platform was used in conjunction with a heat-induced epitope retrieval pro-gram using an epitope retrieval solution (2, ER2; Leica Microsytems Inc.) at pH 9.0. Antibodies to both human CD31 (clone JC70A at a 1:150 dilution; to confirm presence on tumor vessels) and to human B7-H3 (AF1027, at 1:200 dilution; R&D Systems) were used on the same automated platform. Slides were imaged using a digital slide scanner (Nanozoomer). All immunohistochemically stained sections were analyzed by a dedicated breast pathologist. B7-H3 expression on tumor-associated vascular endothelial cells was analyzed using adjacent CD31-stained slices for anatomical guidance to determine presence of tumor vessels. Immunostaining of vessels was scored using a 4-point grading scale: 0=no staining; 1=weak; 2=moderate; and 3=strong vessel staining. Vessel staining was further analyzed for percentage positive vessels using a 5-point grading scale: 0=no positive staining vessels; 1=1%-10%; 2=10%-33%; 3=33%-66%; and 4=66%-100% of positive staining vessels. The results obtained by these two scores were then multiplied together yielding a single value as described (Loos et al. (2009) BMC Cancer 9:463). In addition, microvessel density (MVD) was calculated on all sections using standard techniques (Weidner et al. (1995) Cancer Res. Treat 36:169-180).

B7-H3 Expression on MS1 Cells and Flow Chamber Experiments

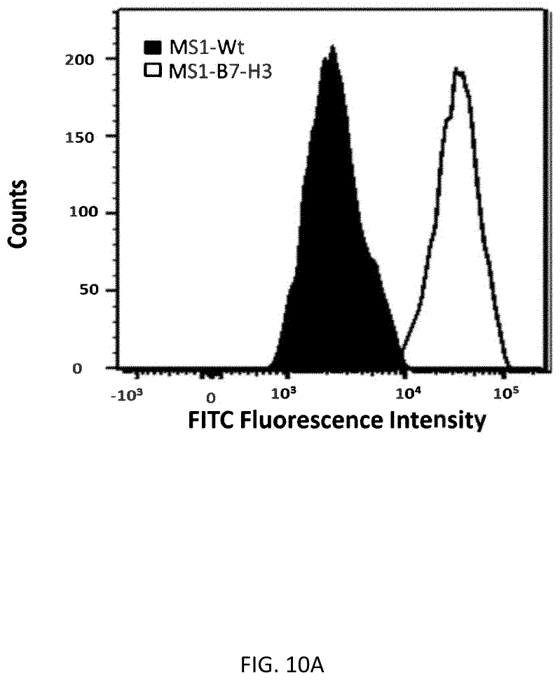

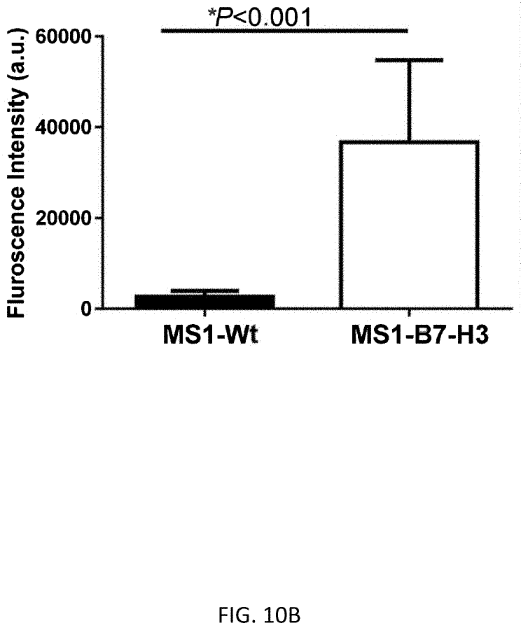

B7-H3 expression levels of wild type MS1 and MS1-B7-H3 cells were assessed by flow cytometry as follows: One million MS1-wt and MS1-B7-H3 cells were washed in phosphate-buffered serum (PBS) and incubated with biotinylated rabbit anti-mouse B7-H3 primary antibody (at 1:100; Abcam) for 1 hour at room temperature. This was followed by incubation with streptavidin-conjugated Fluorescein isothiocyanate (FITC) (1:200; Abcam) for 30 minutes on ice. Expression levels of B7-H3 on the cell surfaces of wild type MS1 and MS1-B7-H3 cells were analyzed with FACSCalibur (Becton Dickinson, San Jose, Calif.) and geometric mean fluorescence intensity was determined using FlowJo software (Stanford University, Stanford, Calif.). MS1-B7-H3 showed significantly increased B7-H3 expression (p<0.001) compared to wild type MS1 (FIG. 10).

One million MS1-wt and MS1-B7-H3 cells were then grown on coated (Sigmacote; Sigma, St Louis, Mo.) glass microscope slides for 24 hours and mounted on a parallel plate flow chamber (GlycoTech, Rockville, Md.). A syringe infusion and withdrawal pump (Genie Plus; Kent Scientific, Torrington, Conn.) was used to maintain the flow rate of 0.6 mL/minute, corresponding to a wall shear stress rate of 100 sec⁻¹ (Jain (1988) Cancer Res 48:2641-2658). The following order was maintained for running each type of microbubble suspension: PBS for 2 minutes, 5×10⁷ of either MB_{B7-H3} or MB_{Control} in PBS for 4 minutes, and 2-minute rinse with PBS. The slides were then removed from the flow chamber apparatus, rinsed briefly in PBS to remove residual microbubbles, and wet mounted with a coverslip for immediate imaging with a phase-contrast bright-field microscope (Axiovert 25; Carl Zeiss, Thomwood, N.Y.; original magnification, ×100). Images were obtained with a digital camera (AxioCam, Bernried, Germany). To further assess binding specificity of MB_{B7-H3} to the target B7-H3, blocking experiments were performed. For this purpose, cells were incubated with 60 μg/mL purified rat-anti-mouse B7-H3 monoclonal antibody (eBioscience, San Diego, Calif.) for 30 minutes at 37° C. to block the receptor prior to mounting the slides on the flow chamber apparatus. Microbubbles can be directly visualized as small, rounded structures and were considered to be attached to cells when there was direct contact with the cells without free floating. Triplicate runs were performed for each cell lines and microbubble type. Five randomly selected optical fields of view per slide were used for subsequent quantification of the number of bound microbubbles per cell.

Cell Culture Experiments

Wild-type MS1 (MS1_{wt}; ATCC) vascular endothelial cells were transfected with B7-H3 expression vector using Lipofectamine 2000 to generate stable MS1 clones (MS1_{B7-H3}) and were maintained in culture under sterile conditions in a 5% CO₂-humidified atmosphere at 37° C. in DMEM and supplemented with 10% FBS and 100 U/mL penicillin and 100 mg/mL streptomycin. Cells were harvested by using trypsinization at 70% to 80% confluence. Routine morphologic analysis under microscope and growth curve analysis were performed to ensure consistent growth properties and authentication according to the ATCC cell line verification test recommendations. The expression of B7-H3 in transfected cells was tested by immunofluorescence imaging with anti-B7-H3 antibody.

Preparation of Targeted and Control Microbubbles

Commercially available preclinical streptavidin-coated, perfluorocarbon containing lipid-shelled microbubbles (VisualSonics, Toronto, Canada) were used to generate B7-H3-targeted microbubbles (MB_{B7-H3}) and control microbubbles (MB_{Control}). Each vial of lyophilized streptavidin coated microbubbles was suspended in 1 mL of sterile saline (0.9% sodium chloride) according to manufacturer's protocol. The mean diameter of microbubbles, as assessed by a cell counter and sizer (Multisizer II Coulter Counter; Beckman Coulter, Fullerton, Calif.), was 1.5±0.1 μm (range, 1-2 μm). Two types of targeted MBs were prepared by using streptavidin-biotin binding chemistry: Microbubbbles targeted to mouse B7-H3 (MB_{B7-H3}), and control MBs targeted with an isotype-matched control immunoglobulin G antibody (MB_{Control}). For this purpose, 6 pg of the following antibodies were incubated with 5×10⁷ MBs for 10 minutes at room temperature: (a) biotinylated rat anti-mouse B7-H3 antibodies (eBiosciences, San Diego, Calif.) and (b) biotinylated isotype-matched control immunoglobulin G antibodies (eBioscience, San Diego, Calif.). The antibody concentration was selected based on previous studies (Deshpande et al. (2011) Radiology 258:804-811. Lindner et al. (2001) Circulation 104:2107-2112, Deshpande et al. (2012) Radiology 262:172-180, Foygel et al. (2013) Gastroenterology 145:885-894) Non-bound antibodies were removed by washing in phosphate buffered saline (PBS). The number of antibodies binding to the microbubble surface was determined by fluorescence-activated cell sorter analysis (FACS) after incubating the targeted microbubbles with fluorescein-conjugated anti-biotin antibody (Jackson ImmunoResearch laboratories, 1:200) as described previously (Deshpande et al., supra). The mean number of attached antibodies per square micrometer of microbubble surface was approximately 7600 for both types of bubbles.

Flow Chamber Experiments

Binding specificity of MB_{B7-H3} to the target B7-H3 was first assessed in cell culture experiments under flow shear stress conditions simulating flow in blood capillaries by using a flow chamber experimental set-up.

Transgenic Mouse Model

All procedures involving the use of laboratory animals were approved by the Institutional Administrative Panel on Laboratory Animal Care. The well-established transgenic mouse model of breast cancer (FVB/N-Tg(MMTV-PyMT)634Mul) was used for all imaging experiments (Bachawal et al., supra; Guy et al. (1992) Mol. Cell Biol. 12:954-961). Breast tissue from control litter mates and normal mammary glands from transgenic mice were used as control normal tissue.

B7-H3-Targeted Contrast-Enhanced Ultrasound Imaging of Mice

Imaging Protocol.

Mammary glands of transgenic mice bearing tumors (n=146) and normal control glands (n=37) were imaged. All mice were kept anesthetized with 2% isoflurane in oxygen at 2 L/min on a heated stage. Targeted contrast-enhanced ultrasound imaging was performed in contrast mode using a dedicated small-animal high resolution ultrasound imaging system (Vevo 2100; VisualSonics, Toronto, Canada). Images were acquired in a transverse plane using a high-resolution transducer (MS250; center frequency, 18 MHz, lateral and axial resolution of 165 μm and 75 μm, respectively; focal length, 8 mm; transmit power, 10%; mechanical index, 0.2; dynamic range, 40 dB). The transducer was fixed on a railing system to maintain the acoustic focus at the center of the mammary gland at the level of the largest transverse cross section. The same imaging settings were used in all imaging experiments.

To differentiate the acoustic signal owing to adherent microbubbles and the signal from freely circulating microbubbles in the bloodstream, principles of ultrasound-induced microbubble destruction and replenishment were used (Lindner et al. (2000) Circulation 102:2745-2750, Kiessling et al. (2012) Curr. Pharm. Des. 18:2184-2199, Foster et al. (2000) Cancer Metastasis Rev. 19:13113-13118). For this purpose, 5×10⁷ MB_{B7-H3} (100 μL) were injected intravenously via tail vein and allowed to attach to B7-H3 on the neovasculature. Four minutes later, 200 imaging frames were captured over a 15-second period to obtain imaging signal from adherent and freely circulating microbubbles. This was followed by a 1-second continuous high power destructive pulse of 3.7 MPa (transmit power, 100%; mechanical index, 0.63) which destroyed all microbubbles within the beam of elevation. Ten seconds after the destruction pulse, another 200 imaging frames were acquired to capture the signal from the influx of freely circulating microbubbles. If more than one injection was performed in one animal, a waiting time of at least 30 minutes was allowed for microbubbles to clear from the previous injection (Bzyl et al. (2011) Eur. Radiol. 21:1988-1995, Pochon et al. (2010) Invest Radiol 45:89-95, Willmann et al. (2008) Radiology 249:212-219, Streeter et al. (2013) Theranostics 3:93-98).

Assessment of Binding Specificity of B7-H3-Targeted Microbubbles In Vivo.

To confirm binding specificity of MB_{B7-H3} to B7-H3 expressed on the tumor neovasculature in transgenic mice, an intra-animal comparison of ultrasound imaging signal following intravenous injection of both 5×10⁷ MB_{B7-H3} and 5×10⁷ MB_{Control} in the same session was performed. For this purpose, mammary glands with breast cancer (n=10) were imaged using both MB_{B7-H3} and MB_{Control} in random order to minimize any bias from the injection order, and injections were separated by at least 30 minutes waiting time to allow clearance of microbubbles from previous injections (Pochon et al. (2010) Invest Radiol 45:89-95). To further confirm binding specificity of MB_{B7-H3} to B7-H3 in the same mice, targeted ultrasound imaging using MB_{B7-H3} was repeated 5 hours after intravenous injection of 125 mg purified rat anti-mouse B7-H3 antibody (eBiosciences) to block B7-H3 receptor sites in vivo.

Data Analysis of In Vivo Imaging Datasets.

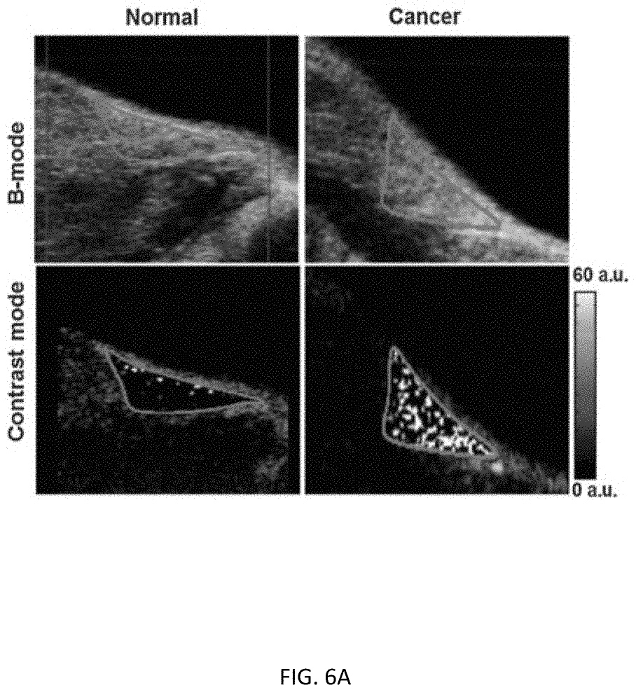

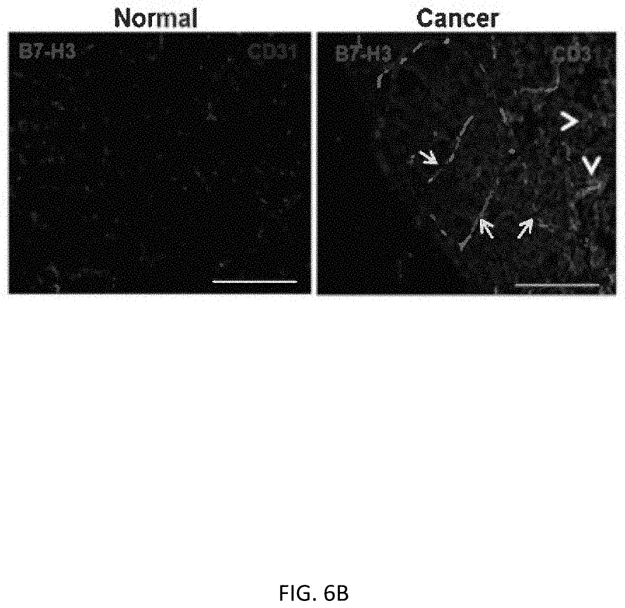

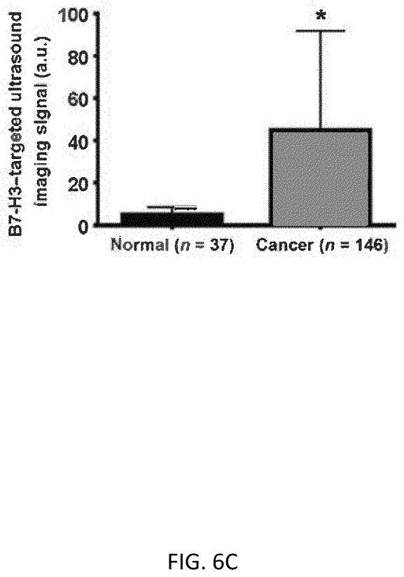

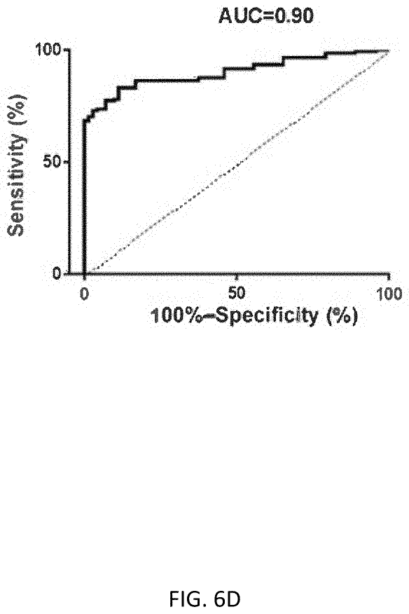

Imaging datasets of all mice were analyzed offline in random order using a dedicated workstation with commercially available software (Vevo 2100, Visualsonics). Analysis was performed in a blinded fashion by one of the authors. Because the transgenic mice used in this study can develop cancer as early as 4 weeks of age and morphologic changes for these early invasive cancers are not visible on conventional B-mode ultrasound imaging (FIG. 6; Lin et al. (2003) Am. J. Pathol. 163:2113-2126), this author was blinded to the mammary gland pathology (normal or cancer). The reader was also blinded to the microbubble type (MB_{B7-H3} or MBControl). Regions of interest (ROI) were drawn over the mammary glands and the magnitude of imaging signal (expressed in arbitrary units, a.u.) from attached microbubbles was assessed by calculating an average for pre- and postdestruction imaging signals and subtracting the average postdestruction signal from the average predestruction signal as described previously (Bzyl et al. (2011) Eur. Radiol. 21:1988-1995, Pochon et al. (2010) Invest. Radiol. 45:89-95, Willmann et al. (2008) Radiology 246:508-518).

Ex Vivo Analysis of Mammary Glands from Transgenic Mice

All mammary glands were excised immediately after imaging to allow direct correlation between in vivo imaging results and ex vivo findings. Before isolating the mammary glands, the imaging plane (position of ultrasound transducer) was marked on the skin of the mammary gland using a tissue marking dye (Cancer Diagnostics, Inc., Morrisville, N.C.) to ensure that the imaged plane was correlated with about the same plane and oriented in the same way on ex vivo analysis. The center of the mammary gland corresponding to the approximate imaging plane was processed for both quantitative immunofluorescence analysis (embedding medium: optimal cutting temperature (OCT) compound) and histopathological analysis (embedding medium: paraffin).

Quantitative Immunofluorescence

Frozen tissue sections (10-20 μm) were rinsed with phosphate-buffered saline (PBS) for 5 minutes to remove the OCT media; this was followed by permeabilization with 0.5% Triton in PBS for 15 minutes and subsequent blocking with 5% BSA, 5% goat and donkey serum in PBS for 1 hour at room temperature. Sections were then co-incubated with rat anti-mouse B7-H3 and rabbit anti-mouse CD31 antibodies overnight at 4° C. at a dilution of 1:50 and 1:100, respectively, and were visualized by using AlexaFluor-488 conjugated goat anti-rabbit or AlexaFluor-546 anti-rat secondary antibodies, respectively (Invitrogen, Grand Island, N.Y.). Double staining for B7-H3 and CD31 was performed to confirm expression of B7-H3 on CD31-positive tumor vascular endothelial cells in mice. The fluorescent images were acquired with a confocal microscope (LSM 510 Meta confocal microscope, Carl Ziess, Germany) at a magnification of 200× using the center plane in a z-stack. To correlate in vivo imaging signal with ex-vivo expression levels of B7-H3, fluorescence intensity of B7-H3 expression on vascular endothelial cells was analyzed and quantified using ImageJ software. Furthermore, MVD analysis was performed using a standardized protocol (Weidner et al. (1995) Breast Cancer Res. Treat. 36:169-180). In brief, the total number of vessels was summed for at least 3 randomly selected fields of view covering the whole tumor section for each tumor slice, and MVD was calculated as the average number of vessels per mm².

Histopathological Analysis

Formalin-fixed and paraffin-embedded sections (5 μm) of all mammary glands were stained with Hematoxylin and Eosin (H&E) and histological diagnosis of breast cancer versus normal tissue was rendered by one breast pathologist blinded to the ultrasound imaging findings. Briefly, a normal mammary gland was comprised of a few ductal epithelial cells surrounded by fat tissue. Cancer was defined as extensive proliferation of breast tissue into a solid mass with little or no fatty tissue in the mammary gland (Cardiff(2001) Microsc. Res. Tech. 52:224-230).

Statistical Analysis

All data were expressed as mean±SD. The Mann-Whitney test was used to show statistical differences in B7-H3 expression in various subtypes in patient samples. Receiver operating curves (ROC) were used to determine the diagnostic accuracy of B7-H3 in differentiating normal, benign and premalignant versus malignant subtypes. The nonparametric Wilcoxon rank sum test was used to compare the binding specificities of MB_{B7-H3} versus MB_{Control} in MS1 cells with and without B7-H3 expression in cell culture experiments and for comparison between in vivo imaging signal after administration of MB_{B7-H3} versus MB_{Control} and following in vivo blocking with anti-B7-H3 antibody. Continuous measurements of the ultrasound signal, ex vivo expression levels of B7-H3, and microvessel density across normal and breast cancer histologies were also tested by the nonparametric Wilcoxon rank sum test. Spearman's correlation coefficients between pairs of variables of interest were estimated and the confidence intervals were constructed based on Fisher's transformation. All statistical analysis was performed using R (2.10.1) software. A P value of less than 0.05 was considered indicative of a significant difference.

Results

Validation of B7-H3 Expression in Human Breast Tissues

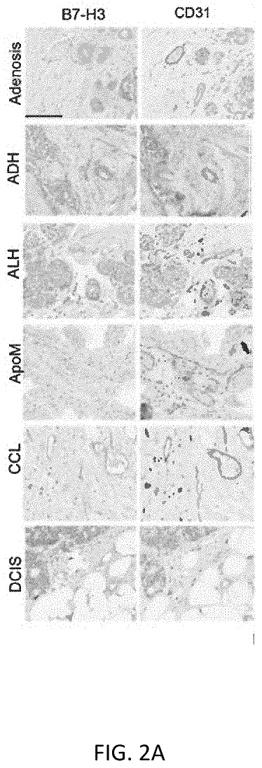

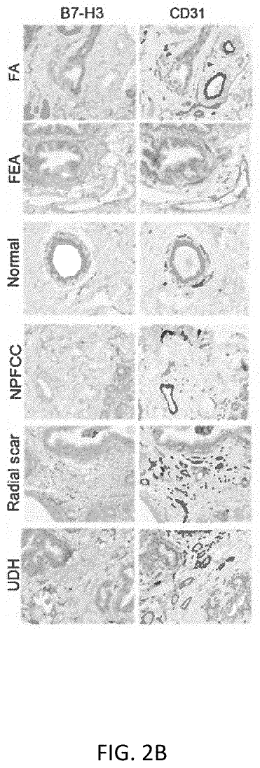

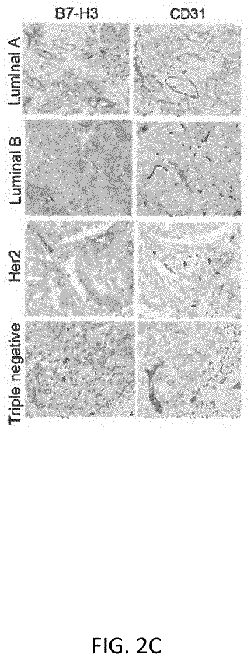

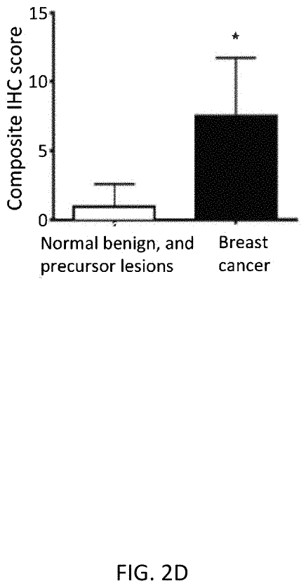

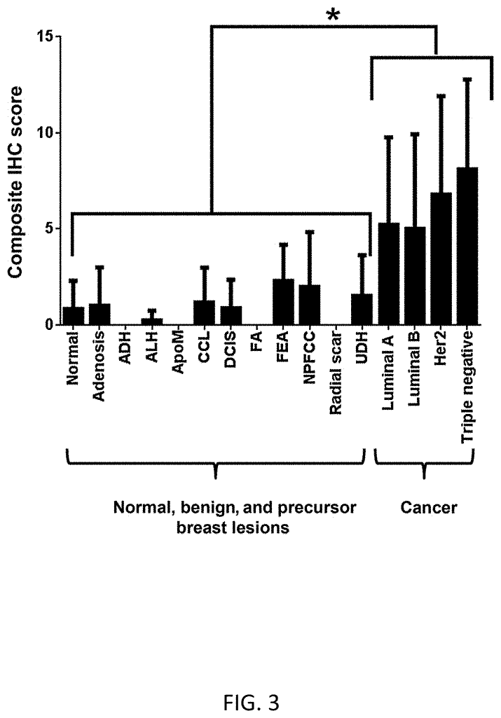

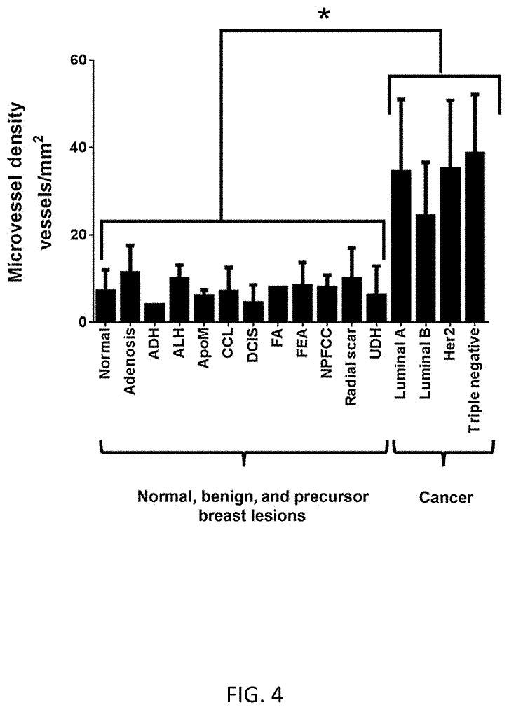

To assess B7-H3 expression in breast cancer-associated neo-vasculature in humans, IHC analysis was performed on breast tissues from a total of 248 women with normal breast tissue (n=47), 11 different benign and precursor breast pathologies (n=100), and four different subtypes of breast cancer (n=101; Table 1). B7-H3 expression was detected on the cell membrane and within the cytoplasm of tumor epithelial cells, on fibroblast-like cells within the stroma, as well as on membranes of vascular endothelial cells. Because of the vascular restriction of the ultrasound molecular contrast agent, only vascular staining (guided by vascular marker CD31 staining) was quantified. In 209 samples processed into a breast TMA, B7-H3 expression was significantly (P<0.001) higher in breast cancer (mean composite score, 7.7) compared with normal tissue, benign, and precursor breast lesions (mean composite score, 1.3; FIG. 2). Individual composite scores for all benign and malignant subtypes are shown in FIG. 3. A detailed summary of B7-H3 staining intensities and percent positive vessels in all normal, benign, premalignant, and malignant human breast tissues is provided in Table 2. MVD was also significantly (P<0.001) increased in breast cancer versus normal, benign, and precursor breast lesions (FIG. 4).

Considering a composite score of 4 or higher as positive staining, overall 88 of 101 breast cancer, 17 of 100 benign lesions, and 6 of 47 normal tissues stained positive. Receiver operating characteristic (ROC) analysis indicated that B7-H3 neovascular immunostaining could distinguish breast cancer from normal tissue, benign, and precursor lesions with an area under the ROC curve (AUC) of 0.90 (95% confidence intervals; CI, 0.86-0.94).

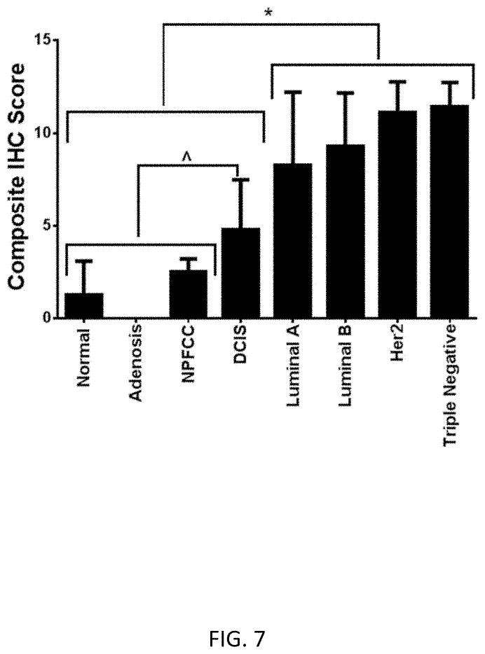

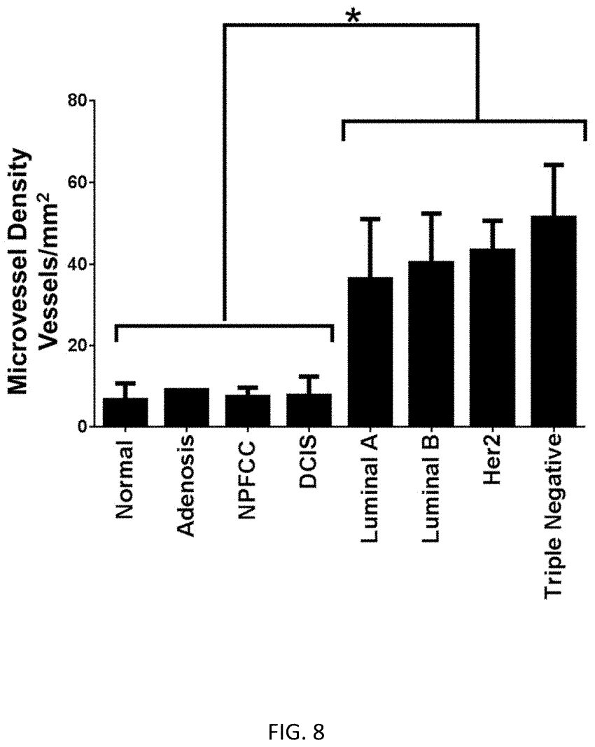

Because TMA represents only very small tissue samples of the various histologies, a subanalysis of an additional 39 whole-tissue samples of breast cancer was performed containing more representative amounts of respective tumor tissues and using the noncancerous surrounding tissue as intra-individual benign controls. In these samples, the mean composite IHC score of malignant lesions (mean composite IHC score, 9.79) was significantly (P<0.001) higher compared with normal tissue, benign, and precursor breast lesions (mean composite IHC score, 1.67; FIG. 7 and Table 3). Considering a composite score of 4 or higher as positive staining, 39 of 39 breast cancer, 5 of 9 benign lesions, and 3 of 30 normal tissues stained positive. This corresponds to an AUC of 0.96 (95% CI, 0.92-0.99) in differentiating cancer versus normal, benign, and precursor lesions. Similarly, the MVD was significantly (P<0.001) increased in breast cancer versus normal tissue, benign, and precursor lesions (FIG. 8).

Flow Chamber Experiments

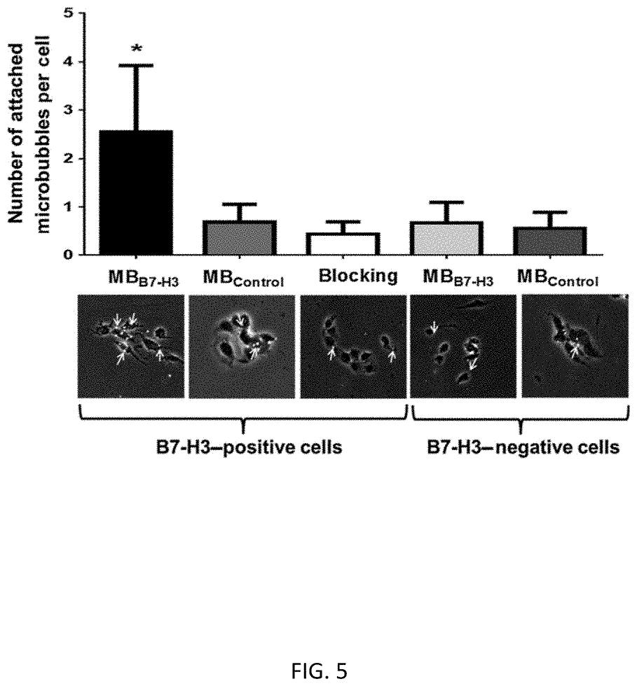

Microbubbles targeted to B7-H3 (MB_{B7-H3}) and control non-targeted microbubbles (MB_{Control}) were synthesized and binding specificity to B7-H3 was first tested in cell culture experiments. FIG. 5 illustrates binding of both MB_{B7-H3} and MB_{Control} to B7-H3-positive and -negative mouse endothelial cells under flow shear stress conditions in a flow chamber. Average number of MB_{B7-H3} attached per cell was significantly higher (P<0.001) on B7-H3-positive compared with negative cells. Blocking of the B7-H3 receptors with anti-B7-H3 antibodies resulted in significantly reduced (P<0.001) binding of MB_{B7-H3} to B7-H3-positive cells, confirming binding specificity of MB_{B7-H3} to B7-H3. There was only minimal nonspecific binding of MB_{Control} to B7-H3-positive cells compared with MB_{B7-H3} (P<0.001).

B7-H3-Targeted Contrast-Enhanced Ultrasound Imaging in Transgenic Mice

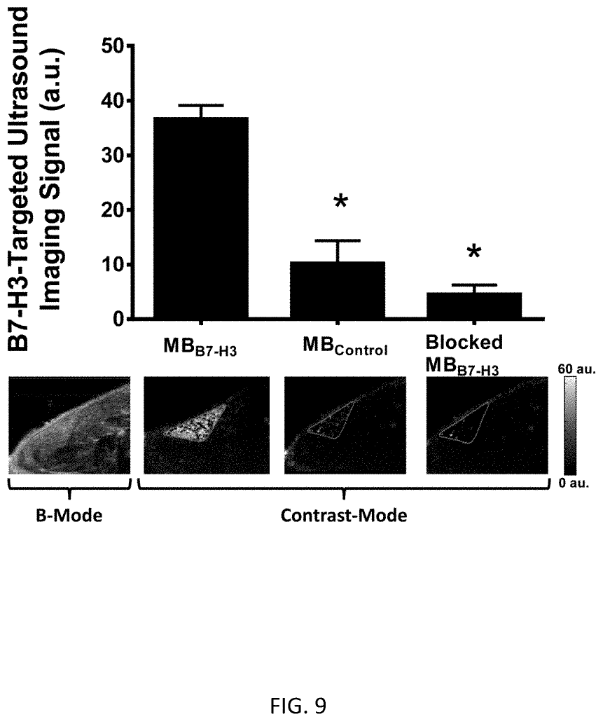

Binding specificity of MB_{B7-H3} to murine B7-H3 was first tested in 10 breast tumors in transgenic mice. In vivo ultrasound imaging signal obtained from MB_{B7-H3} (36.6±7.9 a.u.) was significantly higher (P<0.001) compared with the signal from MB_{Control} (8.4±3.6 a.u.). Furthermore, in vivo B7-H3-targeted ultrasound molecular imaging signal was significantly reduced (4.2±1.6 a.u.; P<0.001) following administration of blocking anti-B7-H3 antibodies, further confirming in vivo binding specificity of MB_{B7-H3} to the imaging target B7-H3 (FIG. 9). We then studied whether ultrasound using B7-H3-targeted contrast microbubbles allows imaging of B7-H3 expression in vivo in 146 mammary glands bearing breast cancer and 37 normal mammary glands. The imaging signal in breast cancer following injection of MB_{B7-H3} (49.4±5.3 a.u.) was significantly higher (P<0.001) in breast cancer than in normal breast tissue (5.0±0.5 a.u.; FIG. 6).

Ex Vivo Analysis

Similar to the human staining, B7-H3 expression was observed both on the tumor neovasculature and on tumor epithelial cells in mice (FIG. 6B). B7-H3 expression on breast cancer-associated neovasculature was significantly (P<0.001) higher (mean intensity, 53±28 a.u.) compared with normal breast tissue (mean intensity, 1.7±1.1 a.u.). Ex vivo B7-H3 expression levels as assessed on quantitative immunofluorescence correlated well (R²=0.77, P<0.001) with in vivo B7-H3-targeted ultrasound imaging signal. MVD was also significantly (P<0.001) higher in breast cancer (mean, 28±16 vessels/mm²) compared with normal mammary tissue (mean, 3±4 vessels/mm²).

Discussion

Our IHC analysis of normal and a broad spectrum of different benign, premalignant, and malignant breast pathologies in women undergoing surgical resection or biopsy show that vascular endothelial cell expression of B7-H3 allows differentiation of breast cancer from benign entities with high diagnostic accuracy. Ultrasound molecular imaging signal in transgenic mice using B7-H3-targeted contrast microbubbles is substantially higher in breast cancer versus normal breast tissue.

In patients with dense breast tissues, ultrasound is currently being explored as a complementary imaging modality to screening mammography for breast cancer detection (Scheel et al. (2015) Am. J. Obstet. Gynecol. 212:9-17). Ultrasound is advantageous because it is widely available, cost-effective, does not expose patients to ionizing radiation, and allows real-time guided biopsy of sonographically detected lesions, if needed.

General limitations of ultrasound as a screening tool, such as long imaging times and operator dependency, are already being addressed by the introduction of commercially available automated whole-breast ultrasound imaging systems that allow a time- and cost-efficient as well as more standardized acquisition and interpretation of breast ultrasound exams (Giuliano et al. (2013) Clin. Imaging 37:480-486). In recent years, molecularly targeted ultrasound contrast agents have been developed to improve diagnostic accuracy of ultrasound in earlier detection of cancer such as pancreatic cancer (Foygel et al., supra; Pysz et al. (2015) Radiology 274:790-799), ovarian (Lutz et al. (2014) Clin. Cancer Res. 20:1313-1322), and breast cancer (Bachawal et al., supra; Bzyl et al., supra). To allow differentiation of cancer from noncancerous tissue using ultrasound and molecularly targeted contrast microbubbles, imaging targets have to be differentially expressed on the neovasculature of cancer compared with vessels in noncancerous tissue. Therefore, the goals of our study were, first, to explore whether a new potential molecular imaging target, B7-H3, is differentially expressed on the neovasculature of human breast cancer and, second, to assess binding specificity of a new B7-H3-targeted ultrasound contrast microbubble both in cell culture and in vivo.

B7-H3, a member of the B7 family of immunoregulators, was first identified on human dendritic cells and activated T cells (Wang et al. (2014) Int. J. Cancer 134:2764-2771, Vigdorovich et al. (2013) Structure 21:707-717). Recently, B7-H3 expression has been shown in several cancer types, including acute leukemia, gastric, pancreatic, renal, liver, lung, bone, colon, prostate, ovarian, endometrial, and breast cancers (Dai et al. (2014) Oncol. Rep. 32:2086-2092, Zhao et al. (2013) Int. J. Mol. Med. 31:283-291, Crispen et al. (2008) Clin. Cancer Res. 14:5150-5157, Wang et al. (2014) Cancer Invest. 32:262-271, Wang et al. (2013) PLoS ONE 8:e70689, Roth et al. (2007) Cancer Res. 67:7893-7900, Zang et al. (2010) Mod. Pathol. 23:1104-1112, Brunner et al. (2012) Gynecol. Oncol. 124:105-111, Maeda et al. (2014) Ann. Surg. Oncol. 21 Suppl. 4:S546-554, Chen et al. (2013) Exp. Cell Res. 319:96-102, Hu et al. (2015) Hematology 20:187-195, Bin et al. (2014) J. Surg. Res. 188:396-403, Arigami et al. (2010) Ann. Surg. 252:1044-1051). However, its role in immune response, including tumor immunity of different cancer types, remains unclear and controversial (Wang et al., supra; Nygren et al. (2011) Front. Biosci. 3:989-993, Loos et al. (2010) Clin. Dev. Immunol. 2010:683875). Both T-cell costimulatory and inhibitory functions have been shown in various cancer types and B7-H3 expression has been correlated with both favorable and poor prognosis in patients with cancer (Wang et al., supra; Dai et al., supra; Wu et al. (2006) World J. Gastroenterol. 12:457-459). For example, in human gastric adenocarcinomas, B7-H3 expression was associated with prolonged patient survival compared with receptor-negative tumors (Wu et al., supra). In contrast, recent studies showed that B7-H3 tumor expression may be a predictor of poor prognosis and increased risk for metastasis in other cancers such as renal, colon, breast, and ovarian cancers (Crispen et al., supra; Zang et al. (2010) Pathol. 23:1104-1112, Maeda et al. (2014) Ann. Surg. Oncol. 21 Suppl. 4:S546-554, Bin et al. (2014) J. Surg. Res. 188:396-403). In women with breast cancer, tumor expression of B7-H3 was suggested as a predictor of early regional lymph node metastases (Arigami et al. (2010) Ann. Surg. 252:1044-1051, Liu et al. (2013) Mol. Med. Rep. 7:134-138), advanced stage disease (Liu et al., supra), and overall worsened prognosis (Maeda et al., supra). Whether B7-H3 is expressed on the neovasculature of breast cancer and whether it can be used as new molecular imaging target for breast cancer with ultrasound remains unclear.

In 248 patient samples including normal, 11 different benign and precursor breast pathologies, and four subtypes of breast cancer, processed both in a TMA and as whole tissue samples, we demonstrated that B7-H3 is overexpressed on breast cancer neovasculature compared with normal, benign, and precursor breast pathologies, using a composite IHC score of both staining intensity and percentage of positively staining vessels. Considering a composite score of 4 or more (out of a maximum of 12) as positive staining, B7-H3 allowed differentiation of breast cancer from normal, benign, and precursor lesions with high diagnostic accuracy. Because TMAs only represent a very small sample of tumor or benign tissues, an IHC subanalysis of 39 whole-tissue breast cancer samples was also performed. In this subgroup, all breast cancer types showed positive B7-H3 staining on the neovasculature. Peri-tumoral breast tissues served as intra-individual controls and confirmed substantially less staining in normal, benign, or precursor breast lesions associated with breast cancer.

After validation of B7-H3 as a potential vascular molecular imaging target for human breast cancer detection, B7-H3-targeted microbubbles were designed and tested both in cell culture experiments and in vivo. Flow chamber experiments simulating shear stress flow in tumor vessels confirmed binding specificity of B7-H3-targeted microbubbles to their molecular target. This was further confirmed in breast cancer imaging experiments in vivo, which showed substantially higher ultrasound molecular imaging signal in breast cancer following intravenous injection of B7-H3-targeted microbubbles compared with control microbubbles in intra-animal comparison experiments in the same breast cancers. Quantitative immunofluorescence of excised murine mammary tissues further confirmed vascular expression of B7-H3 with excellent quantitative correlation between in vivo imaging signal and ex vivo expression levels of B7-H3. These results suggest that B7-H3-targeted ultrasound molecular imaging should be further developed as a noninvasive, relatively inexpensive imaging approach for breast cancer detection in patients.

We acknowledge the following limitations of our study. For this proof-of-principle imaging study in mice, we used biotin-streptavidin binding chemistry and commercially available antibodies to generate B7-H3-targeted microbubbles. These were not intended for clinical use and ongoing experiments explore the design of clinical grade contrast microbubbles targeted at B7-H3 using techniques described previously (Pochon et al., supra; Pysz et al. (2010) Radiology 256:519-527). Also, due to the small dimensions of murine breast tissues in the z-plane, we chose to scan mice in two-dimensional planes only in our study. Automatic whole breast scanners are now available in the clinic (Giuliano et al., supra), which will facilitate future translation of volumetric ultrasound molecular imaging for screening purposes in patients. Finally, although we assessed breast cancer-associated B7-H3 vascular endothelial cell expression in human tissue samples in a broad spectrum of benign and malignant breast lesions by IHC, B7-H3-targeted ultrasound molecular imaging was only tested in normal and invasive breast cancer in vivo. To the best of our knowledge, no mouse models are available that harbor the spectrum of all the benign diseases tested in the human samples in our study, which would allow modeling the diagnostic accuracy of B7-H3-targeted ultrasound molecular imaging in preclinical studies before translating this approach into the clinic. Therefore, future clinical studies using clinical grade B7-H3-targeted contrast microbubbles are warranted to both confirm our human IHC staining results and to assess diagnostic accuracy of ultrasound molecular imaging in detecting and characterizing breast cancer in patients.

In conclusion, our results suggest that B7-H3 is differentially expressed on the neovasculature of breast cancer compared with normal breast tissue and multiple benign breast pathologies in women undergoing surgical resection or biopsy. Ultrasound molecular imaging signal using contrast microbubbles targeted at B7-H3 is substantially increased in breast cancer versus normal breast tissue in transgenic mice. Future work toward clinical translation will develop clinical grade contrast agents targeted at B7-H3 that will eventually help in improving the diagnostic accuracy of ultrasound screening exams in detection and characterization of breast lesions in women with dense breast tissue.

TABLE 1
Histologic subtype and sample size. Summary of various
breast pathologies collected and analyzed by IHC

| Histology | Subtype | n |
|---|---|---|
| Normal breast tissue | N.A. | 47 |
| Benign and Precursor Breast Lesions | Adenosis | 4 |
| | ADH | 1 |
| | ALH | 4 |
| | ApoM | 4 |
| | CCL | 57 |
| | DCIS | 10 |
| | FA | 1 |
| | FEA | 7 |
| | NPFCC | 2 |
| | Radial scar | 2 |
| | UDH | 8 |
| Breast cancer | Luminal A | 45 |
| | Luminal B | 16 |
| | Her2 | 19 |
| | Triple negative | 21 |

Abbreviations: ADH, atypical ductal hyperplasia; ALH, atypical lobular hyperplasia; ApoM, apocrine metaplasia; CCL, columnar cell lesion; DCIS, ductal carcinoma in situ; FA, fibroadenoma; FEA, flat epithelial atypia; NPFCC, nonproliferative fibrocystic changes; UDH, usual ductal hyperplasia; Her2, human epidermal growth factor receptor type 2 positive cancer; Luminal A, estrogen receptor and/or progesterone receptor-positive cancer; Luminal B, estrogen receptor- and/or progesterone receptor-positive and Her2-positive cancer; Triple negative, estrogen, progesterone, and Her2-negative breast cancer.

TABLE 2
Summary of B7-H3 Immunohistochemistry Scores in Human Tissue Samples.
Summary of B7-H3 staining intensities and percent positive vessels of
all normal, benign premalignant and malignant human breast samples.

| | Staining | | % | | | Staining | | % | | | Staining | |
|---|---|---|---|---|---|---|---|---|---|---|---|---|
| | Intensity | | Positive | | | Intensity | | Positive | | | Intensity | |
| Histology | (n) | | Vessels | n | Histology | (n) | | Vessels | n | Histology | (n) | |
| Normal | 0 | (32) | 0 | 32 | Adenosis | 0 | (3) | 0 | 3 | ADH | 0 | (1) |
| | 1 | (13) | 1-10 | 2 | | 1 | (1) | 1-10 | — | | 1 | (0) |
| | | | 10-33 | — | | | | 10-33 | — | | | |
| | | | 33-66 | 6 | | | | 33-66 | | | | |
| | | | >66 | 5 | | | | >66 | 1 | | | |
| | >1 | (2) | 1-10 | 1 | | >1 | (0) | 1-10 | — | | >1 | (0) |
| | | | 10-33 | — | | | | 10-33 | — | | | |
| | | | 33-66 | — | | | | 33-66 | — | | | |
| | | | >66 | 1 | | | | >66 | — | | | |
| ApoM | 0 | (4) | 0 | 4 | CCL | 0 | (36) | 0 | 36 | DCIS | 0 | (6) |
| | 1 | (0) | 1-10 | — | | 1 | (20) | 1-10 | — | | 1 | (3) |
| | | | 10-33 | — | | | | 10-33 | 6 | | | |
| | | | 33-66 | — | | | | 33-66 | 5 | | | |
| | | | >66 | — | | | | >66 | 9 | | | |
| | >1 | (0) | 1-10 | — | | >1 | (1) | 1-10 | — | | >1 | (1) |
| | | | 10-33 | — | | | | 10-33 | — | | | |
| | | | 33-66 | | | | | 33-66 | — | | | |
| | | | >66 | — | | | | >66 | 1 | | | |
| FEA | 0 | (3) | 0 | 3 | NPFCC | 0 | (1) | 0 | 1 | Radial | 0 | (2) |
| | 1 | (4) | 1-10 | 1 | | 1 | (1) | 1-10 | — | Scar | 1 | (0) |
| | | | 10-33 | — | | | | 10-33 | — | | | |
| | | | 33-66 | — | | | | 33-66 | — | | | |
| | | | >66 | 3 | | | | >66 | 1 | | | |
| | >1 | (0) | 1-10 | — | | >1 | (0) | 1-10 | — | | >1 | (0) |
| | | | 10-33 | — | | | | 10-33 | — | | | |
| | | | 33-66 | — | | | | 33-66 | — | | | |
| | | | >66 | — | | | | >66 | — | | | |
| Luminal A | 0 | (7) | 0 | 7 | Luminal B | 0 | (0) | 0 | 0 | | 0 | (1) |
| | 1 | (11) | 1-10 | 0 | | 1 | (2) | 1-10 | — | | 1 | (2) |

| | | 10-33 | — | | | 10-33 | — | |
|---|---|---|---|---|---|---|---|---|
| | | 33-66 | 4 | | | 33-66 | | |
| | | >66 | 7 | | | >66 | 2 | Her2 |
| >1 | (27) | 1-10 | — | >1 | (14) | 1-10 | — | |

| | | | | | |
|---|---|---|---|---|---|
| 10-33 | — | 10-33 | 1 | >1 | (16) |

| | | | |
|---|---|---|---|
| 33-66 | — | 33-66 | 1 |
| >66 | 27 | >66 | 12 |

| | | | | Staining | | | |
|---|---|---|---|---|---|---|---|
| | % Positive | | | Intensity | | % Positive | |
| Histology | Vessels | n | Histology | (n) | | Vessels | n |
| Normal | 0 | 1 | ALH | 0 | (3) | 0 | 3 |
| | 1-10 | — | | 1 | (1) | 1-10 | 1 |
| | 10-33 | — | | | | 10-33 | — |
| | 33-66 | | | | | 33-66 | — |
| | >66 | — | | | | >66 | — |
| | 1-10 | — | | >1 | (0) | 1-10 | — |
| | 10-33 | — | | | | 10-33 | — |
| | 33-66 | — | | | | 33-66 | — |
| | >66 | — | | | | >66 | — |
| ApoM | 0 | 6 | | 0 | (1) | 0 | 1 |
| | 1-10 | — | | 1 | (0) | 1-10 | — |
| | 10-33 | — | | | | 10-33 | — |
| | 33-66 | 3 | | | | 33-66 | — |
| | >66 | — | | | | >66 | — |
| | 1-10 | — | | >1 | (0) | 1-10 | — |
| | 10-33 | — | | | | 10-33 | — |
| | 33-66 | — | | | | 33-66 | — |
| | >66 | 1 | | | | >66 | — |
| FEA | 0 | 2 | UDH | 0 | (4) | 0 | 4 |
| | 1-10 | — | | 1 | (3) | 1-10 | 1 |
| | 10-33 | — | | | | 10-33 | — |
| | 33-66 | — | | | | 33-66 | 2 |
| | >66 | — | | | | >66 | — |
| | 1-10 | — | | >1 | (1) | 1-10 | — |
| | 10-33 | — | | | | 10-33 | — |
| | 33-66 | — | | | | 33-66 | 1 |
| | >66 | — | | | | >66 | — |
| Luminal A | 0 | 1 | Triple | 0 | (0) | 0 | — |
| | 1-10 | 1 | Negative | 1 | (2) | 1-10 | — |
| | 10-33 | — | | | | 10-33 | — |
| | 33-66 | | | | | 33-66 | — |
| | >66 | 1 | | | | >66 | 2 |
| | 1-10 | — | | >1 | (19) | 1-10 | — |
| | 10-33 | — | | | | 10-33 | — |
| | 33-66 | 2 | | | | 33-66 | 2 |
| | >66 | 14 | | | | >66 | 17 |

TABLE 3
Sub-analysis of B7-H3 Immunohistochemistry Scores in Cancer and Surrounding Normal, Benign and Precursor Lesions.
Summary of B7-H3 staining intensities and percent positive vessels analyzed in a subgroup of 39 cancer samples.
Whole tissue samples including the surrounding normal, benign, and precursor lesions were analyzed.

| | Stain- | | | | | Stain- | | | | | Stain- | | | | | Stain- | | | |
|---|---|---|---|---|---|---|---|---|---|---|---|---|---|---|---|---|---|---|---|
| | ing | | | | | ing | | | | | ing | | | | | ing | | | |
| | Inten- | | % | | | Inten- | | % | | | Inten- | | % | | | Inten- | | % | |
| Histol- | sity | | Positive | | Histol- | sity | | Positive | | Histol- | sity | | Positive | | Histol- | sity | | Positive | |
| ogy | (n) | | Vessels | n | ogy | (n) | | Vessels | n | ogy | (n) | | Vessels | n | ogy | (n) | | Vessels | n |
| Normal | 0 | (18) | 0 | 18 | s | 0 | (1) | 0 | 1 | NPFCC | 0 | (0) | 0 | — | DCIS | 0 | (0) | 0 | — |
| | 1 | (12) | 1-10 | — | | 1 | (0) | 1-10 | — | | 1 | (2) | 1-10 | — | | 1 | (2) | 1-10 | — |
| | | | 10-33 | 8 | | | | 10-33 | — | | | | 10-33 | 1 | | | | 10-33 | 1 |
| | | | 33-66 | 1 | | | | 33-66 | — | | | | 33-66 | | | | | 33-66 | 1 |
| | | | >66 | 3 | | | | >66 | — | | | | >66 | 1 | | | | >66 | — |
| | >1 | (0) | 0 | — | | >1 | (0) | 0 | — | | >1 | (0) | 0 | — | | >1 | (4) | 0 | — |
| | | | 1-10 | — | | | | 1-10 | — | | | | 1-10 | — | | | | 1-10 | — |
| | | | 10-33 | — | | | | 10-33 | — | | | | 10-33 | — | | | | 10-33 | — |
| | | | 33-66 | — | | | | 33-66 | — | | | | 33-66 | — | | | | 33-66 | 1 |
| | | | >66 | — | | | | >66 | — | | | | >66 | — | | | | >66 | 3 |
| Luminal A | 0 | (0) | 0 | — | | 0 | (0) | 0 | — | Her2 | 0 | (0) | 0 | — | Triple | 0 | (0) | 0 | — |
| | 1 | (3) | 1-10 | — | | 1 | (0) | 1-10 | — | | 1 | (0) | 1-10 | — | Negative | 1 | (0) | 1-10 | — |
| | | | 10-33 | — | | | | 10-33 | — | | | | 10-33 | — | | | | 10-33 | — |
| | | | 33-66 | — | | | | 33-66 | — | | | | 33-66 | — | | | | 33-66 | — |
| | | | >66 | 3 | | | | >66 | — | | | | >66 | — | | | | >66 | — |
| | >1 | (10) | 0 | — | | >1 | (13) | 0 | — | | >1 | (8) | 0 | — | | >1 | (5) | 0 | — |

| | | | | | | | |
|---|---|---|---|---|---|---|---|
| 1-10 | — | 1-10 | — | 1-10 | — | 1-10 | — |
| 10-33 | — | 10-33 | 1 | 10-33 | — | 10-33 | — |
| 33-66 | 1 | 33-66 | — | 33-66 | 1 | 33-66 | — |
| >66 | 7 | >66 | 9 | >66 | 7 | >66 | 5 |

While the preferred embodiments of the invention have been illustrated and described, it will be appreciated that various changes can be made therein without departing from the spirit and scope of the invention.

What is claimed is:

1. A method of detecting breast cancer, the method comprising:
a) administering a detectably effective amount of a B7 homolog 3 (B7-H3)-targeted imaging agent to a patient suspected or at risk of having breast cancer, under conditions wherein the B7-H3-targeted imaging agent binds to B7-H3 on breast tumor neovasculature, breast tumor epithelium, cancerous cells, or precancerous lesions, if present, in the patient; and
b) detecting the B7-H3-targeted imaging agent bound to the breast tumor neovasculature, breast tumor epithelium, cancerous cells, or precancerous lesions, if present, by imaging breast tissue of the patient.

2. The method of claim 1, wherein said imaging is performed using ultrasound imaging (UI), positron emission tomography (PET), single photon emission computed tomography (SPECT), magnetic resonance imaging (MRI), computed tomography (CT), optical imaging (OI), photoacoustic imaging (PI), fluoroscopy, or fluorescence imaging.

3. The method of claim 1, wherein the breast cancer is luminal A breast cancer, luminal B breast cancer, triple negative breast cancer, or Her2-positive breast cancer.

4. The method of claim 1, wherein detection of a precancerous lesion indicates the patient is at risk of developing breast cancer.

5. The method of claim 1, wherein the patient is human.

6. The method of claim 1, wherein the B7-H3-targeted imaging agent comprises a B7-H3-targeting agent conjugated to a diagnostic agent.

7. The method of claim 6, wherein the B7-H3-targeting agent is an anti-B7-H3 antibody, peptide, peptoid, aptamer, or small molecule ligand that selectively binds to B7-H3.

8. The method claim 7, wherein the anti-B7-H3 antibody is a monoclonal antibody, a polyclonal antibody, a chimeric antibody, a recombinant fragment of an antibody, an Fab fragment, an Fab′ fragment, an F(ab′)₂ fragment, an Fᵥ fragment, or an scFᵥ fragment.

9. The method of claim 1, wherein the diagnostic agent is a contrast agent or a photoactive agent.

10. The method of claim 9, wherein the contrast agent is an ultrasound contrast agent, a magnetic resonance imaging (MRI) contrast agent, or a radiocontrast agent.

11. The method of claim 10, wherein the ultrasound contrast agent is a microbubble.

12. The method of claim 1, wherein the diagnostic agent comprises a detectable label.

13. The method of claim 12, wherein the detectable label is a fluorescent label, a radioactive isotopic label, a non-radioactive isotopic label, a chemiluminescent label, a bioluminescent label, a paramagnetic ion, or an enzyme.

14. The method of claim 13, wherein the fluorescent label is selected from the group consisting of a fluorescein derivative, a rhodamine derivative, a coumarin derivative, a cyanine derivative, an acridine derivative, a squaraine derivative, a naphthalene derivative, an oxadiazol derivative, an anthracene derivative, a pyrene derivative, an oxazine derivative, an arylmethine derivative, a tetrapyrrole derivative, and a fluorescent protein.

15. The method of claim 14, wherein the fluorescent protein is selected from the group consisting of green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, rsCherry, and rsCherryRev.

16. The method of claim 13, wherein the isotopic label is selected from the group consisting of ³H, ²H, ¹²⁰I, ¹²³I, ¹²⁴I, ¹²⁵I, ¹³¹I, ³⁵S, ¹¹C, ¹³C, ¹⁴C, ³²P, ¹⁵N, ¹³N, ¹¹⁰In, ¹¹¹In, ¹⁷⁷Lu, ¹⁸F, ⁵²Fe, ⁶²Cu, ⁶⁴Cu, ⁶⁷Cu, ⁶⁷Ga, ⁶⁸Ga, ⁸⁶Y, ⁹⁰Y, ⁸⁹Zr, ^{94m}Tc, ⁹⁴Tc, ^{99m}Tc, ¹⁵⁴Gd, ¹⁵⁵Gd, ¹⁵⁶Gd, ¹⁵⁷Gd, ¹⁵⁸Gd, ¹⁵O, ¹⁸⁶Re, ¹⁸⁸Re, ⁵¹M, ^{52m}Mn, ⁵⁵Co, ⁷²As, ⁷⁵Br, ⁷⁶Br, ^{82m}Rb, and ⁸³Sr.

17. The method of claim 13, wherein the radioactive isotopic label comprises a radionuclide selected from the group consisting of a gamma-emitter, a beta-emitter, and a positron-emitter.

18. The method of claim 13, wherein the paramagnetic ion is selected from the group consisting of chromium (III), manganese (II), iron (III), iron (II), cobalt (II), nickel (II), copper (II), neodymium (III), samarium (III), ytterbium (III), gadolinium (in), vanadium (II), terbium (III), dysprosium (III), holmium (Ill) and erbium (III).

19. The method of claim 6, wherein the B7-H3-targeted imaging agent is further conjugated to an anti-cancer therapeutic agent.

20. The method of claim 19, wherein the anti-cancer therapeutic agent is selected from the group consisting of a cytotoxic agent, a drug, a toxin, a nuclease, a hormone, an immunomodulator, a pro-apoptotic agent, an anti-angiogenic agent, a boron compound, a photoactive agent, and a radioisotope.

21. The method of claim 1, further comprising recording at least one image of the breast tissue with a charge-coupled device (CCD) image sensor, a CMOS image sensor, or a digital camera.

22. A method of performing image-guided surgery on breast tissue of a patient having breast cancer, the method comprising:
a) detecting breast cancer according to the method of claim 1; and
b) using at least one image of the breast tissue to determine where resection of cancerous tissue is needed.

23. The method of claim 22, wherein the imaging is performed with a medical imaging device selected from the group consisting of an ultrasound scanner, a magnetic resonance imaging instrument, a radiography system, an X-ray computed tomography (CT) scanner, a computed axial tomography (CAT) scanner, a gamma camera, and a positron emission tomography (PET) scanner.

24. The method of claim 22, wherein the imaging is performed pre-operatively to assist surgical planning.

25. The method of claim 22, wherein the imaging is performed intra-operatively to provide image-guidance during surgery.

26. The method of claim 22, wherein the medical imaging device is a miniaturized medical imaging system.

27. The method of claim 26, wherein the miniaturized medical imaging system comprises a handheld microscope, a laparoscope, an endoscope, or a microendoscope.

28. The method of claim 26, wherein the miniaturized medical imaging system is an intravascular ultrasound (IVUS) imaging device or a fluorescence imaging device.

29. The method of claim 22, wherein the imaging is used for tumor margin delineation or evaluation of completeness of resection.

30. The method of claim 22, wherein the imaging is real-time imaging.

31. A method of imaging breast tissue of a patient suspected or at risk of having breast cancer, the method comprising:
a) contacting breast tissue of the patient with a detectably effective amount of a B7-H3-targeted imaging agent under conditions wherein the B7-H3-targeted imaging agent binds to B7-H3 on any breast tumor neovasculature, breast tumor epithelium, cancerous cells, or precancerous lesions, if present, in the breast tissue; and
b) imaging breast tissue of the patient, wherein detection of increased binding of the B7-H3-targeted imaging agent to the breast tissue of the patient compared to a control indicates that the patient has breast cancer.

32. The method of claim 31, wherein the breast tissue is contacted with the B7-H3-targeted imaging agent in vivo or in vitro.

33. The method of claim 31, wherein said imaging is performed using ultrasound imaging (UI), positron emission tomography (PET), single photon emission computed tomography (SPECT), magnetic resonance imaging (MRI), computed tomography (CT), optical imaging (OI), photoacoustic imaging (PI), fluoroscopy, or fluorescence imaging.

34. A method of monitoring progression of breast cancer in a patient, the method comprising: imaging breast tissue of the patient according to the method of claim 31, wherein a first image is obtained at a first time point and a second image is obtained later at a second time point, wherein detection of increased binding of the B7-H3-targeted imaging agent to the breast tissue of the patient at the second time point compared to the first time point indicates that the patient is worsening, and detection of decreased binding of the B7-H3-targeted imaging agent to the breast tissue of the patient at the second time point compared to the first time point indicates that the patient is improving.

35. The method of claim 34, wherein increased binding of the B7-H3-targeted imaging agent to the breast tissue of the patient is associated with growth of a breast tumor or presence of more breast tumors or lesions at the second time point.

36. A method for evaluating the effect of an agent for treating breast cancer in a patient, the method comprising: imaging breast tissue of the patient according to the method of claim 31 before and after the patient is treated with said agent, wherein detection of increased binding of the B7-H3-targeted imaging agent to the breast tissue of the patient after the patient is treated with said agent compared to before the patient is treated with said agent indicates that the patient is worsening, and decreased binding of the B7-H3-targeted imaging agent to the breast tissue of the patient after the subject is treated with said agent compared to before the patient is treated with said agent indicates that the patient is improving.

37. A method for monitoring the efficacy of a therapy for treating breast cancer in a patient, the method comprising: imaging breast tissue of the patient according to the method of claim 31 before and after the subject undergoes said therapy, wherein detection of increased binding of the B7-H3-targeted imaging agent to the breast tissue of the patient after the patient undergoes said therapy compared to before the patient undergoes said therapy indicates that the patient is worsening, and decreased binding of the B7-H3-targeted imaging agent to the breast tissue of the patient after the patient undergoes said therapy compared to before the patient undergoes said therapy indicates that the patient is improving.

38. A method of treating a patient suspected or at risk of having breast cancer, the method comprising:
a) receiving information regarding whether or not breast cancer was detected in the patient according to the method of claim 1; and
b) administering anti-cancer therapy to the subject if breast cancer was detected in the patient.

39. The method of claim 38, wherein the anti-cancer therapy comprises surgery, radiation therapy, chemotherapy, hormonal therapy, immunotherapy, or biologic therapy, or any combination thereof.

40. A method for diagnosing and treating a patient suspected or at risk of having breast cancer, the method comprising:
a) contacting breast tissue of the patient with a detectably effective amount of a B7-H3-targeted imaging agent under conditions wherein the B7-H3-targeted imaging agent binds to B7-H3 on any breast tumor neovasculature, breast tumor epithelium, cancerous cells, or precancerous lesions, if present, in the breast tissue;
b) diagnosing the patient by imaging breast tissue of the patient, wherein detection of increased binding of the B7-H3-targeted imaging agent to the breast tissue of the patient compared to a control indicates that the patient has breast cancer; and
c) administering anti-cancer therapy to the subject if the patient is diagnosed with breast cancer.

41. The method of claim 40, wherein the anti-cancer therapy comprises surgery, radiation therapy, chemotherapy, hormonal blocking therapy, immunotherapy, or biologic therapy, or any combination thereof.

42. The method of claim 41, wherein the surgery is a mastectomy, a quadrantectomy, or a lumpectomy.

43. The method of claim 41, wherein the chemotherapy comprises administration of cyclophosphamide, doxorubicin, docetaxel, cyclophosphamide, methotrexate, or fluorouracil.

44. The method of claim 41, wherein the hormonal blocking therapy comprises administration of a therapeutically effective amount of tamoxifen, anastrozole, or letrozole.

45. The method of claim 41, wherein the immunotherapy comprises administration of an anti-HER2 receptor antibody.

46. The method of claim 45, wherein the anti-HER2 receptor antibody is Trastuzumab.

47. A B7-H3-targeted imaging agent comprising an anti-B7-H3 antibody conjugated to a microbubble.

48. A kit comprising the B7-H3-targeted imaging agent of claim 47 and instructions for using the kit for detecting or imaging breast cancer.
